(19) United States
(12) Patent Application Publication
(10) Pub. No.: US 2025/0195463 A1
(43) Pub. Date: Jun. 19, 2025

(54) ORGANOMETALLIC COMPLEX, CONTROLLED-RELEASE MULTI-FUNCTIONAL DRUG, PHARMACEUTICAL COMPOSITION, PROCESSES FOR THE PREPARATION THEREOF AND THEIR USE

(71) Applicant: FACULDADE DE CIÊNCIAS DA UNIVERSIDADE DE LISBOA, LISBOA (PT)

(72) Inventors: Tânia Sofia FERREIRA MORAIS, ERICEIRA (PT); Maria Helena ANSELMO VIEGAS GARCIA, LISBOA (PT); João Miguel FRANCO MACHADO

(21) Appl. No.: 18/846,557

(22) PCT Filed: Mar. 22, 2023
(86) PCT No.: PCT/IB2023/052803

§ 371 Date: Sep. 12, 2024

(30) Foreign Application Priority Data
Apr. 13, 2022 (PT) 117911

(51) Int. Cl. A61K 31/295; A61K 31/555; A61K 47/64; A61P 35/00; C07F 17/02

(52) U.S. Cl. CPC A61K 31/295; A61K 31/555; A61K 47/64; A61P 35/00; C07F 17/02

(57) ABSTRACT

The present invention provides an organometallic complex, which can be incorporated in a targeting smart delivery system to form a controlled-release multi-functional drug. In particular, the present invention relates to an organometallic complex of general formula I and to a controlled-release multi-functional drug of general formula II and the processes for the preparation thereof.

The invention further relates to pharmaceutical compositions comprising the organometallic complex or the controlled-release multi-functional drug and the use thereof for efficient, site-specific drug delivery, more specifically, as a diagnosis and/or therapeutic agent in cancer therapy and/or cancer prevention, independently of the stage of the disease.

### TECHNICAL FIELD

The present invention relates to the field of medicine, more specifically, to the fields of medical imaging, diagnosis, and pharmaceutical therapy and provides an organometallic complex and a controlled-release multi-functional drug, as well as the processes for the preparation thereof.

It is also an object of the present invention a pharmaceutical composition comprising such organometallic complex or controlled-release multi-functional drug and their uses for efficient, site-specific drug delivery in cancer therapy, cancer prevention and/or cancer diagnosis.

### BACKGROUND ART

Cancer remains an uncurable disease with continuous rising of incidence, prevalence, morbidity, and mortality over the years, being currently the second leading cause of death worldwide. The clinically available anticancer treatments show limited efficacy and undesirable side effects, mostly due to low selectivity for cancer cells over healthy tissues.

Among them, metallodrugs play a key role in current treatment and diagnosis of cancer, being part of the majority of in-use chemotherapeutic regimens. Despite being well established in clinical practice with positive outcomes, particularly against early diagnosed and primary cancers, the broader use of metallodrugs is impaired by their severe side effects, the intrinsic or acquired resistance to them, and their inability to reach cancer metastases.

Metal complexes have been intensively studied as potential anticancer agents and are considered an emerging class of metallodrug candidates. Several metal complexes have been reported to show in vitro and in vivo antitumoral, antimetastatic and/or antiangiogenic properties against several types of cancers. Desirable advantages of some of these metal complexes include higher efficacy, intrinsic selectivity, lower toxicity, alternative modes of action, capacity to overcome resistance to conventional therapies, and/or synergistic/additive effects with another chemotherapeutics.

Particularly, numerous half-sandwich (“piano-stool”) organometallic complexes have shown very promising anticancer and drug-like properties. As example, documents WO 2007/128158 A1 and WO 2013/136296 A2 both disclose several families of half-sandwich organometallic complexes for the treatment of cancer and metastases, and to inhibit mechanisms of drug resistance, yet these patent applications do not encompass vehicles for the selective delivery of the organometallic complexes to the target.

Indeed, despite the enhanced anticancer properties of the inorganic and organometallic complexes reported in literature, most of them still face the same drawbacks as conventional chemotherapeutics. Their inadequate intrinsic selectivity towards cancer and metastases over healthy tissues impairs further drug development and clinical use. To overcome this limitation, an increasingly number of vectorizing systems have been developed for selective delivery of the metal complexes into cancer cells.

This approach relies on tethering the complexes (active cargo) to a carrier that has high affinity and selectivity for a cancer-related target (delivery agent/targeting unit), and thus allows the specific accumulation of the anticancer metal complex in the cancer site. The metal cargo is often tethered to the delivery agent (such as peptide, protein, antibody, polymer, sugar, hormone, biomolecule, aptamer, cyclodextrin, dendrimer, liposome, micelle, among others) by conjugation, linkage, adsorption, entrapping, or encapsulation. Few pertinent examples are listed below.

WO 2016/087932 A2 discloses half-sandwich organometallic complexes with macromolecular ligands, such as polymers, for treatment or prevention of cancer. The macromolecular ligand coordinated to the metal center ensures passive tumor targeting by means of enhanced permeability and retention effect.

JP2005047821 A encompass half-sandwich complexes of a radioactive metal with carbonyl ligands, conjugated to a tumor targeting-unit, such as peptides, proteins, or antibodies, for cancer diagnosis or therapy.

WO 2015081117 A2 discloses complexes of transition metals with peptides for targeted delivery to cancer cells expressing tumor markers of choice.

CN 108484726 A and CN 108558950 A are two examples of patent applications that encompass the conjugation of an inorganic ruthenium complex to a delivery peptide which are said to be useful in cancer therapy.

Franco Machado, et al. (2020) describes three organometallic half-sandwich ruthenium peptide-conjugates for potential application in treatment of breast cancer. The delivery peptides used are specific for the fibroblast growth factor receptor (FGFR) overexpressed in some types of breast cancer. Conjugation of said peptides to the organometallic complexes conferred them higher selectivity for FGFR(+)-breast cancer cells over FGFR(−)-breast cancer cells, but also resulted in a loss of anticancer activity.

Other examples of the delivery of half-sandwich organometallic complexes with peptides include the studies performed by Splith, K. et al. (2010); Hu, W. et al. (2012); Hoyer, J. et al. (2012) and NDongo, H. W. P. et al. (2008), which reported the conjugation of half-sandwich rhenium or manganese complexes with carbonyl ligands to delivery peptides, either unspecific cell penetrating peptides for enhanced cell uptake or tumor-specific peptides for active targeting.

Nonetheless, despite the improved selectivity observed for the above stated metallodrug delivery systems, as well as for other systems described in literature (e.g. Franco Machado, J.; Correia, J. D. G. and Morais, T. S. (2021)), in generally they still face problems of reduced anticancer activity or inadequate release of the metal complex as major drawbacks. Indeed, as a result, none of the cited metallodrug delivery systems have achieved clinical trials, not to mention actual therapies.

Therefore, there is the need for improved delivery systems capable of releasing the metal complexes in a site- and time-specific manner to surpass the referred limitations. Smart delivery systems that only release the drug in its active form at the cancer site in response to tumor-related stimuli (e.g. pH, glutathione, oxygen, reactive oxygen species, enzymes, among others) might thus be a plausible solution. This emerging therapeutical approach is considered very promising, although few examples have been described yet given its novelty.

Incorporating approved drugs into smart delivery systems for cancer therapy has been already reported, however these systems mainly encompass organic drugs. As example, Jin, Y. et al. (2015)) describes the conjugation of doxorubicin to a delivery peptide specific of the cancer-related protein LAPTM4B by means of hydrazone bond, rendering the system responsiveness to tumoral pH and thus controlled release in cancer cells.

US 2012171227 A1 discloses prodrugs for cancer therapy or diagnosis, composed of a therapeutic agent and one or more functional groups, such as a delivery agent, a diagnosis agent, or a triggering agent for controlled delivery and release of the therapeutic agent at the cancer site upon a specific stimulus, yet this patent application does not encompass transition metal complexes as the therapeutic agent.

Zhao, Z. et al. (2019) and Zhao, Z. et al. (2018) are examples of works where transition metal complexes have been selectively delivered to cancer cells using pH-responsive smart delivery systems. However, in these papers, the anticancer metal cargo is an inorganic ruthenium complex that is released from the targeting unit (a peptide or a vitamin) by exchange of the ligand that is conjugated to the targeting unit by two water molecules, with subsequent modification of the complex coordination sphere.

Very few cases of stimuli-dependent smart delivery systems capable of releasing the active metal complex in the same form as prior to its incorporation in the delivery system have been reported. Wang, T. et al. (2015) describes the potential use of a glutathione-responsive prodrug for cancer photodynamic therapy, based on a luminescent inorganic ruthenium complex conjugated through a disulphide bond to a hormone specific of the cancer-related somatostatin receptor. Analogously, Lv, G. et al. (2019) reports the smart delivery of an organometallic para-cymene-ruthenium complex to cancer cells by using a hormone specific of the progesterone receptor as targeting unit and a disulphide bond as linker for controlled released.

Moreover, Splith, K. et al. (2010) describes a family of organometallic complexes of manganese with carbonyl ligands conjugated to cell penetrating peptides through a protease cleavable sequence for their delivery and controlled release into cancer cells. However, in all the cases herein cited, the chemical structures of the metal complexes reported are considerably distinct from those of the organometallic metallodrugs encompassed in the present invention.

Aiming to fill the gap in the prior art cited and to offer novel therapeutic approaches to fight cancer, it is thus object of the present invention to provide processes for the preparation, pharmaceutical compositions, and diagnostic/medical use of organometallic complexes, which can be incorporated in a targeting smart delivery system and form a controlled-release multi-functional drug capable of site-specific delivery into cancer cells and metastases. The controlled-release multi-functional drug is composed of three units: an organometallic complex, a cancer-specific stimuli sensitive-linking moiety, and a targeting unit.

The targeting unit corresponds to a delivery agent that recognizes selectively and with high affinity a specific physiological, structural and/or chemical feature of cancer cells/tissues and therefore allows selective accumulation in the tumour site, endowing the system with tumour tracking-ability, both against primary cancers and cancer metastases. The organometallic complex has high cytotoxicity and favourable anticancer properties, being thus capable of eradicating the primary cancer and/or its metastases; and also contains in its chemical structure one or more chemical groups that upon reaction with the targeting unit originate the linking moiety. In turn, the linking moiety is stable under healthy physiological conditions, but sensitive to the tumour microenvironment and/or cancer-specific stimuli. Altogether, the three units compose a controlled-release multi-functional drug endowed with controlled self-dissociation ability, being inert during biodistribution and innocuous to the healthy tissues but rendered responsive to the tumour microenvironment related stimuli where the active organometallic complex is free to act. Therefore, these drugs are expected to show fewer toxicity than current available anticancer treatments due to the specific accumulation and controlled release of the active organometallic complex only at the cancer site, which also boosts its therapeutic efficacy while reducing the adverse effects in number and in severity.

The results gathered indicate that the controlled-release multi-functional drugs object of the present invention are capable of selectively deliver the highly cytotoxic organometallic complexes in their active form into cancer cells overexpressing cancer-specific receptors, in a controlled way induced by a stimulus arising from the tumour microenvironment. These are important and advantageous features required for any drug intended to be used in precise and personalized medicine.

The new class of organometallic complexes and controlled-release multi-functional drugs, as well as pharmaceutical compositions comprising thereof, aim thus at supplying a treatment with added therapeutic value comparatively to current clinically available options for cancer prevention and/or cancer diagnosis and/or cancer therapy, as well as for the prevention, diagnosis and/or therapy of cancer metastases.

These and other advantages of the present invention will become more apparent and will be better understood from the following description, claims, and appended drawings. Additional objectives, advantages, and characteristics will also become apparent to those ordinarily skilled in the art by the analysis of the description or the practice of the invention.

Throughout the description and the claims of the present invention, the words and expressions “comprise”, “include”, “preferential”, “preferable”, “such as”, “for instance”, “as example”, their variations and similar should not be understood as excluding other technical features or components.

### SUMMARY OF INVENTION

The present invention is directed to provide ways to overcome the limitations of the current treatment, prevention and/or identification of cancer referred to in the state of the art. In particular, the present invention provides an organometallic complex and a controlled-release multi-functional drug for the delivery of said organometallic complex to a site of interest in a living body, aiming preventing or treating diseases associated with cancer.

The present invention discloses, in a first aspect, an organometallic complex having the general Formula I

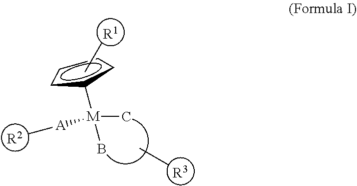

or a pharmaceutically acceptable salt or solvate thereof, wherein:
M is a heteroatom selected from the group consisting of ruthenium, iron, rhodium, cobalt, iridium, osmium, molybdenum, manganese, rhenium, and technetium;
the cyclopentadienyl ring comprises at least one substituent R₁, wherein each substituent R₁ is bonded to any carbon atom of said cyclopentadienyl ring;
A is a monodentate ligand optionally bonded to at least one substituent R₂, the monodentate ligand being preferably selected from the group consisting of heteroaromatic ligands, phosphanes, —CO, —NO, nitriles, isonitriles, a metal complex or a molecule of biological interest;
   wherein the heteroatom in the heteroaromatic ligand is selected from the group consisting of O, S, P, N or Se;
   wherein the R₂ is present where the rules of valency permit;
   wherein the metal complex is selected from the group consisting of inorganic or organometallic complexes with a transition metal selected from the group consisting of ruthenium, iron, rhodium, cobalt, iridium, osmium, molybdenum, manganese, rhenium, and technetium;
   wherein the molecule of biological interest is selected from the group consisting of vitamins, hormones, amino acids, sugars, and peptides; BC together represent a bidentate ligand optionally bonded to at least one substituent R₃, the bidentate ligand being preferably selected from the group consisting of heteroaromatic ligands, diphosphanes, a metal complex or a bidentate molecule of biological interest
   wherein the heteroatoms in the heteroaromatic ligands are the same heteroatoms or different heteroatoms, the heteroatoms being independently selected from group consisting of O, S, P, N or Se;
   wherein the R₃ is present where the rules of valency permit;
   wherein the bidentate molecule of biological interest is selected from the group consisting of vitamins, hormones, amino acids, sugars, and peptides;
   wherein BC is taken together with said heteroatom M to which it is attached to form a 3-12-membered heterocyclyl or 5-12 membered heteroaryl ring, wherein each heterocyclyl or heteroaryl ring optionally contains 1, 2 or 3 additional heteroatoms selected from the group consisting of O, S, P, N or Se; each of R₁, R₂ and R₃ are independently selected from the group consisting of H, C₁-C₆ alkyl, C₁-C₆ aryl, —YCOY′, —YCHO, —YCONHNH₂, —YNHNH₂, —YCOOH, —YCOOY′, —YOCOOH, —YOCOOY′, —YNHCOOH, —YNHCOOY′, —YNHY′, —YONH₂, —YCH(CH₂)ₙ(OH)₂, —YPh(OH)₂, —YSH; —YCH(CH₂)ₙ(SH)₂, —YPh(SH)₂, —YB(OH)₂, —YOB(OH)₂, —YOPO₃H₂, —YOPO₂H₂, —Y(CH₂)ₙX, —YCOX, —YCHCH₂, —YN₃, —Y-imidazole, —YOCOO(CH₂)₂SH, —YNHCOO(CH₂)₂SH and —Y-AA,
   wherein
   Y and Y′ are independently selected from the group consisting of H, C₁-C₆ alkyl and C₁-C₆ aryl; each of said alkyl or aryl being unsubstituted or substituted by carbonyl, amide, ester, ether or thioether;
   n is an integer selected from 0 to 6;
   Ph is a phenyl group;
   X is a halogen atom selected from the group consisting of F, Cl, Br and I;
   the heteroaryl ring is a 5-12 membered heteroaryl ring wherein each heteroaryl ring optionally contains 1, 2 or 3 heteroatoms selected from the group consisting of O, S, P, N or Se; and
   AA is a cis-aconitic anhydride group
with the proviso that at least one of R₁, R₂ or R₃ is not H.

A second aspect of the present invention discloses several processes for the preparation of such organometallic complex.

In a third aspect of the present invention, it is disclosed a controlled-release multi-functional drug having the general Formula II

OC-L-TU (Formula II)
wherein
OC is the organometallic complex as defined in any of claims 1-4, or a pharmaceutically acceptable salt or solvate thereof,
TU is a targeting unit selected from the group consisting of peptide, protein, amino acid, peptidomimetic, antibody, affibody, nanobody, aptamer, antigen, or binding fragments thereof, which are able to selectively bind to receptors or drug targets overexpressed at cancer cell surfaces,
   wherein the targeting unit TU has a R₄ group in its structure, the R₄ group being selected from the group consisting of H, C₁-C₆ alkyl, C₁-C₆ aryl, —YCOY′, —YCHO, —YCONHNH₂, —YNHNH₂, —YCOOH, —YCOOY′, —YOCOOH, —YOCOOY′, —YNHCOOH, —YNHCOOY′, —YNHY′, —YONH₂, —YCH(CH₂)ₙ(OH)₂, —YPh(OH)₂, —YSH; —YCH(CH₂)ₙ(SH)₂, —YPh(SH)₂, —YB(OH)₂, —YOB(OH)₂, —YOPO₃H₂, —YOPO₂H₂, —Y(CH₂)ₙX, —YCOX, —YCHCH₂, —YN₃, —Y-imidazole, —YOCOO(CH₂)₂SH, —YNHCOO(CH₂)₂SH and —Y-AA,
      wherein
      Y and Y′ are independently selected from the group consisting of H, C₁-C₆ alkyl and C₁-C₆ aryl; each of said alkyl or aryl being unsubstituted or substituted by carbonyl, amide, ester, ether or thioether;
      n is an integer selected from 0 to 6;
      Ph is a phenyl group;
      X is a halogen atom selected from the group consisting of F, Cl, Br and I;
      the heteroaryl ring is a 5-12 membered heteroaryl ring wherein each heteroaryl ring optionally contains 1, 2 or 3 heteroatoms selected from the group consisting of oxygen, sulfur, phosphorus, nitrogen and selenium; and
      AA is a cis-aconitic anhydride group;
the organometallic complex OC and the targeting unit TU being bonded via a linking moiety L between the organometallic complex OC and the targeting unit TU;
   wherein the linking moiety L results from the reaction of the R₄ from the targeting unit TU with the R′ from the organometallic complex OC;
   wherein the R′ from organometallic complex OC is any one of R₁, R₂ or R₃;
   wherein at least one linking moiety L and at least one targeting unit TU are bonded in the same R′ or in different R′;
   wherein the linking moiety L is selected from the group consisting of —YC(Y′)═NNH—; —YNHN═C(Y′)—; —YC(Y′)═NNHC(O)—; —YC(O)NHN═C(Y′)—; —YC(Y′)═NC—; —C(Y′)═NCY—; —YPhC(Y′)═NC—; —YC(Y′)═NPh-; —YN═NY′—; —YC(O)NY′—; —C(O)N(Y′)Y—; cis-aconityl; —YC(Y′)═NO—; —C(Y′)═NOY—; —YC(Y′)(OY″)O—; —YC(Y′)(OH)O—; —YC(O)OC—; —YC(OY′)(OY″)OC—; —YOB(OY′)OC—; —YB(OY′)OC—; —YOC(O)OC—; bis-carbonate; para-aminobenzylcarbonate; —YOC(O)NY′—; —OC(O)N(Y′)Y—; para-methoxybenzyl carbamate, —YOC(O)CH₂CH₂SY′—; N-ethoxybenzylimidazole; N-alkoxyalkylimidazole; phosphoramidyl; phosphoramidate; —YSSY′—; disulphide carbamate, —YC(Y′)(SY″)S—; bis-alkylcarbonate dissulphide; bis-alkylcarbonate thioketal; —YSY′—; β-glucuronide; β-galactoside; or trityl,
      wherein
      Y, Y′ and Y″ are independently selected from the group consisting of H, C₁-C₆ alkyl and C₁-C₆ aryl; each of said alkyl or aryl being unsubstituted or substituted by carbonyl, amide, ester, ether or thioether;
      n is an integer selected from 0 to 6;
      Ph is a phenyl group; and
the heteroaryl ring is a 5-12 membered heteroaryl ring wherein each heteroaryl ring optionally contains 1, 2 or 3 heteroatoms selected from the group consisting of oxygen, sulfur, phosphorus, nitrogen and selenium.

A fourth aspect of the present invention discloses a process for the preparation of the controlled-release multi-functional drug.

A fifth aspect of the present invention relates to the use of the organometallic complex and/or the controlled-release multi-functional drug as an antitumoral agent and/or anti-metastatic agents and/or radio sensitizer agent in cancer therapy and/or cancer prevention.

A sixth aspect of the present invention relates to the use of the organometallic complex and/or the controlled-release multi-functional drug as a diagnosis agent and/or theragnostic agent in cancer identification.

In a seventh aspect of the present invention a pharmaceutical composition comprising such organometallic complex or a pharmacologically acceptable salt and/or solvate thereof, or such controlled-release multi-functional drug or a pharmaceutically acceptable salt or solvate thereof and at least one active ingredient and/or pharmaceutically acceptable carrier, excipient, or diluent.

An eighth aspect of the present invention relates to the use of the pharmaceutical composition as an antitumoral agent and/or anti-metastatic agents and/or radio sensitizer agent in cancer therapy and/or diagnosis and/or cancer prevention.

### Technical Problem

The broader use of inorganic and organometallic complexes is impaired by their severe side effects, the intrinsic or acquired resistance to them, and their inability to reach cancer metastases.

Also, their inadequate intrinsic selectivity towards cancer and metastases over healthy tissues impairs further drug development and clinical use.

### Solution to Problem

The present invention provides organometallic complex that has high cytotoxicity and favourable anticancer properties, being thus capable of eradicating the primary cancer and/or its metastases.

When incorporated in a targeting smart delivery system and forming a controlled-release multi-functional drug, the results indicate that the drug is capable of selectively deliver the highly cytotoxic organometallic complexes in their active form into cancer cells overexpressing cancer-specific receptors, in a controlled way induced by a stimulus arising from the tumour microenvironment.

These are important and advantageous features required for any drug intended to be used in precise and personalized medicine.

### Advantageous Effects of Invention

Compared with the prior art, the present invention provides processes for synthesizing an organometallic complex and a controlled-release multi-functional drug, as well as a pharmaceutical composition comprising the same, which are capable of controlled releasing the active organometallic complex only at the tumor/metastases.

The MTT assay showed that the controlled-release multi-functional drug can selectively target FGFR overexpressing tumor cells with lower activity on non-overexpressing FGFR cancer cells, this suggests that these pharmaceutical compositions can be used as novel targeted antitumor medicine for cancers that overexpress receptors.

These and other advantages of the present invention will be clear and better understood from the following description and appended drawings and claims. Nevertheless, the examples and drawings are provided to illustrate the inventive concepts and are not intended to limit the scope of the invention.

### BRIEF DESCRIPTION OF DRAWINGS

In order to promote an understanding of the principles according to the modalities of the present invention, reference will be made to the modalities illustrated in the figures and the language used to describe them.

It should also be understood that there is no intention to limit the scope of the invention to the content of the figures and that modifications to the inventive features illustrated herein, as well as additional applications of the principles and embodiments illustrated, which would normally occur to a person skilled in the art having the possession of this description, are considered within the scope of the claimed invention.

FIG. 1

FIG. 1A-H are graphs showing the chemical stability in organic medium (dimethyl sulfoxide) over 24 h of the complexes 4, 5, 6, 7, 8, 9, 10, 11, respectively.

FIG. 2

FIG. 2A-H are graphs showing the chemical stability in aqueous medium (5% dimethyl sulfoxide/95% Dulbecco's Modified Eagle Medium) over 24 h of the complexes 4, 5, 6, 7, 8, 9, 10, 11, respectively.

FIG. 3

FIG. 3A-B are graphs representing the results of the release of the complex 4 from conjugate RuPC1 at pH 6.8 and pH 7.4, respectively: RP-HPLC chromatograms acquired over 50 h.

FIG. 4

FIG. 4A-B are graphs representing the variation of the relative (%) area under the curve (AUC) of the compounds detected by RP-HPLC during the drug release studies of RuPC1 at pH 6.8 and pH 7.4, respectively, over 50 h.

FIG. 5

FIG. 5 is a dose-response graph representing the cell viability for the complexes 4, 5, 8 and 9, after 48 h of incubation, in the human breast carcinoma cells MDA-MB-231.

FIG. 6

FIG. 6A-D are dose-response graphs representing the cell viability for the conjugate RuPC1, complex 4 and peptide P2, after 48 h of incubation, in the human breast carcinoma cells MDA-MB-231, MCF-7, SK-BR-3 and MDA-MB-134-VI, respectively.

FIG. 7

FIG. 7A-D are dose-response graphs representing the cell viability for the conjugate RuPC1 at pH 6.8 and at pH 7.4, after 48 h of incubation, in the human breast carcinoma cells MDA-MB-231, MCF-7, SK-BR-3 and MDA-MB-134-VI, respectively.

### DESCRIPTION OF EMBODIMENTS

The present invention provides an organometallic complex, which can be incorporated in a targeting smart delivery system to form a controlled-release multi-functional drug. The process for the preparation thereof and their uses are also an object of the present invention. Both organometallic complex and controlled-release multi-functional drug can be formulated into a pharmaceutical composition. Its use is also an object of the present invention.

In one embodiment of the present invention, the organometallic complex has the general Formula I

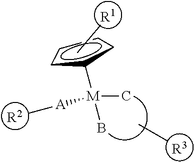

or is a pharmaceutically acceptable salt or solvate thereof,
wherein
M is a heteroatom selected from the group consisting of ruthenium, iron, rhodium, cobalt, iridium, osmium, molybdenum, manganese, rhenium, and technetium;
the cyclopentadienyl ring comprises at least one substituent R₁, wherein each substituent R₁ is bonded to any carbon atom of said cyclopentadienyl ring;
A is a monodentate ligand optionally bonded to at least one substituent R₂, the monodentate ligand being preferably selected from the group consisting of heteroaromatic ligands, phosphanes, —CO, —NO, nitriles, isonitriles, a metal complex or a molecule of biological interest;
   wherein the heteroatom in the heteroaromatic ligand is selected from the group consisting of O, S, P, N or Se;
   wherein the R₂ is present where the rules of valency permit;
   wherein the metal complex is selected from the group consisting of inorganic or organometallic complexes with a transition metal selected from the group consisting of ruthenium, iron, rhodium, cobalt, iridium, osmium, molybdenum, manganese, rhenium, and technetium;
   wherein the molecule of biological interest is selected from the group consisting of vitamins, hormones, amino acids, sugars, and peptides;
BC together represent a bidentate ligand optionally bonded to at least one substituent R₃, the bidentate ligand being preferably selected from the group consisting of heteroaromatic ligands, diphosphanes, a metal complex or a bidentate molecule of biological interest
   wherein the heteroatoms in the heteroaromatic ligands are the same heteroatoms or different heteroatoms, the heteroatoms being independently selected from group consisting of O, S, P, N or Se;
   wherein the R₃ is present where the rules of valency permit;
   wherein the bidentate molecule of biological interest is selected from the group consisting of vitamins, hormones, amino acids, sugars, and peptides;
   wherein BC is taken together with said heteroatom M to which it is attached to form a 3-12-membered heterocyclyl or 5-12 membered heteroaryl ring, wherein each heterocyclyl or heteroaryl ring optionally contains 1, 2 or 3 additional heteroatoms selected from the group consisting of O, S, P, N or Se;
each of R₁, R₂ and R₃ are independently selected from the group consisting of H, C₁-C₆ alkyl, C₁-C₆ aryl, —YCOY′, —YCHO, —YCONHNH₂, —YNHNH₂, —YCOOH, —YCOOY′, —YOCOOH, —YOCOOY′, —YNHCOOH, —YNHCOOY′, —YNHY′, —YONH₂, —YCH(CH₂)ₙ(OH)₂, —YPh(OH)₂, —YSH; —YCH(CH₂)ₙ(SH)₂, —YPh(SH)₂, —YB(OH)₂, —YOB(OH)₂, —YOPO₃H₂, —YOPO₂H₂, —Y(CH₂)ₙX, —YCOX, —YCHCH₂, —YN₃, —Y-imidazole, —YOCOO(CH₂)₂SH, —YNHCOO(CH₂)₂SH and —Y-AA,
   wherein
   Y and Y′ are independently selected from the group consisting of H, C₁-C₆ alkyl and C₁-C₆ aryl; each of said alkyl or aryl being unsubstituted or substituted by carbonyl, amide, ester, ether or thioether;
   n is an integer selected from 0 to 6;
   Ph is a phenyl group;
   X is a halogen atom selected from the group consisting of F, Cl, Br and I;
   the heteroaryl ring is a 5-12 membered heteroaryl ring wherein each heteroaryl ring optionally contains 1, 2 or 3 heteroatoms selected from the group consisting of O, S, P, N or Se; and
   AA is a cis-aconitic anhydride group;
      with the proviso that at least one of R₁, R₂ or R₃ is not H.

In a preferred embodiment of the invention, M is ruthenium.

In another preferred embodiment of the invention, A is a monodentate ligand. More preferably, A is triphenylphosphane and R₂ is H.

In another preferred embodiment of the invention, BC is a bidentate heteroaromatic ligand wherein both heteroatoms are nitrogen. More preferably, BC is 2,2′-bipyridyl; 1,10-phenanthroline or pyrazino[2,3-f][1,10]phenanthroline.

In another preferred embodiment of the invention, each of R₁, R₂ and R₃ are independently selected from the group consisting of H, C₁-C₆ aryl, —YCOY′, —YCONHNH₂ and —YNHNH₂
wherein
Y and Y′ are independently selected from the group consisting of H, C₁-C₆ alkyl and C₁-C₆ aryl; each of said alkyl or aryl being unsubstituted or substituted by carbonyl, amide, ester, ether or thioether; and
the heteroaryl ring is a 5-12 membered heteroaryl ring wherein each heteroaryl ring optionally contains 1, 2 or 3 heteroatoms selected from the group consisting of O, S, P, N or Se; and
   with the proviso that at least one of R₁, R₂ or R₃ is not H.

More preferably, each of R₁ and R₃ are independently selected from the group consisting of —H, —COCH₃, —COCH₂CH₃, —COCH₂Ph, —CONHNH₂, —OH, —OCO₂CH₂CH₃ and —OCO(CH₂)₂COCH₃, with the proviso that at least one of R₁ or R₃ is not H.

In the most preferred embodiment of the invention, the organometallic complex is

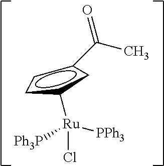

or chlorobis(triphenylphosphane)(η⁵-methyl cyclopentadienylketone) ruthenium(II);

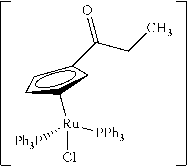

or chlorobis(triphenylphosphane)(f-ethyl cyclopentadienylketone) ruthenium(II);

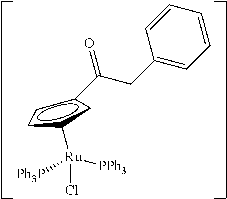

or chlorobis(triphenylphosphane)(f-phenylmethyl cyclopentadienylketone) ruthenium(II);

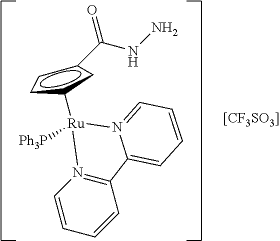

or (2,2′-bipyridyl)(triphenylphosphane)(f-cyclopentadienylhydrazide) ruthenium(II) trifluoromethanesulfonate;

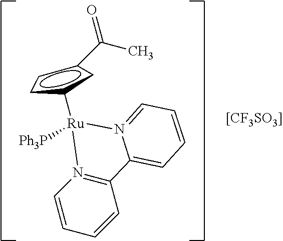

or (2,2′-bipyridyl)(triphenylphosphane)(f-methyl cyclopentadienylketone) ruthenium(II) trifluoromethanesulfonate;

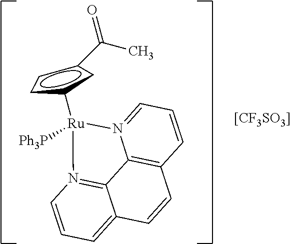

or (1,10-phenanthroline)(triphenylphosphane)(η⁵-methyl cyclopentadienylketone) ruthenium(II) trifluoromethanesulfonate;

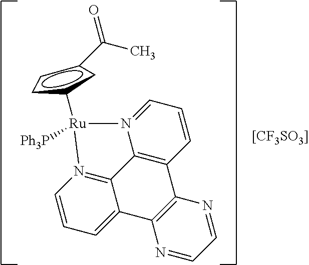

or (pyrazino[2,3-f][1,10]phenanthroline) (triphenylphosphane) (η⁵-methyl cyclopentadienylketone) ruthenium(II) trifluoromethanesulfonate;

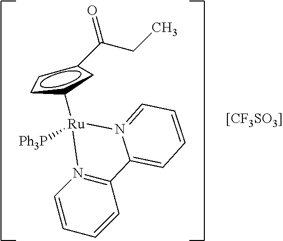

or (2,2′-bipyridyl)(triphenylphosphane)(n-ethyl cyclopentadienylketone) ruthenium(II) trifluoromethanesulfonate;

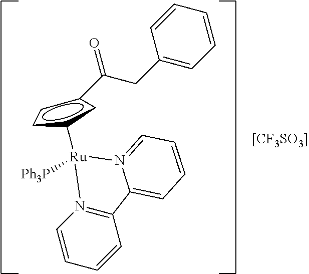

or (2,2′-bipyridyl)(triphenylphosphane)(η⁵-phenylmethyl cyclopentadienylketone) ruthenium(II) trifluoromethanesulfonate;

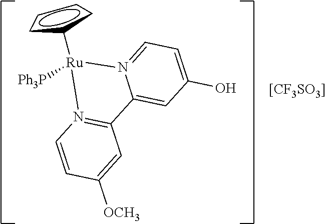

or (4-hydroxy-4′-methoxy-2,2′-bipyridyl)(triphenylphosphane)(η⁵-cyclopentadienyl) ruthenium(II) trifluoromethanesulfonate;

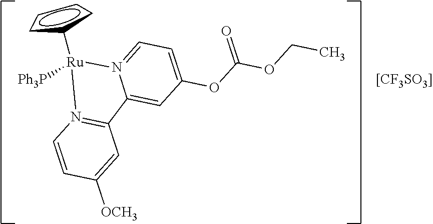

or (4-(ethoxycarbonyl)oxy-4′-methoxy-2,2′-bipyridyl)(triphenylphosphane)(η⁵-cyclopentadienyl) ruthenium(II) trifluoromethanesulfonate; or

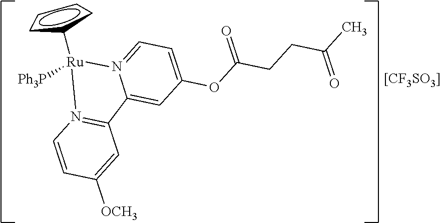

or (4-methoxy-4′-(4-oxopentanoate)-2,2′-bipyridyl)(triphenylphosphane)(η⁵-cyclopentadienyl) ruthenium(II)trifluoromethanesulfonate.

The organometallic complexes of formula I can be cationic or neutral and can be in salt form, whose anions are selected from the group consisting of halides, phosphates, triflates, phenylborates and hexafluorophosphates.

The organometallic complexes of the present invention are air stable and stable in aqueous media for an appropriate period for medicinal purposes and have controlled release profile from the multi-functional drug favorable to the selective delivery of the active organometallic complexes into the tumor/metastases and very relevant antitumor properties.

The present invention further provides, in another aspect, processes for the preparation of the organometallic complexes of formula I as described above, or a pharmaceutically acceptable salt and/or solvate thereof. Said processes are selected among any of the following processes:

Process I—reacting a compound of formula [M(Cp-_{R1})(LL)X] or its solvates or its salts with A-R₂ or BC-R₃ ligands in the presence or in the absence of a halide abstraction reagent selected from the group consisting of AgCF₃SO₃, AgBF₄, AgPF₆, AgNO₂, AgNO₃, AgSO₄, NaCF₃SO₃, NaBF₄, NaPF₆, NaBArF, KCF₃SO₃, NH₄CF₃SO₃ or TlCF₃SO₃.

In process I, Cp is a cyclopentadienyl ring, LL is two A-R₂ ligands; one BC-R₃ ligand; two monodentate ligands; or one bidentate ligand and X is a halide selected from the group consisting of fluoride, chloride, bromide, or iodide.

Process II—comprises the steps of (i) reacting a compound of formula [MLnXn] or its solvates or its salts with Cp-R₁ or ZCp-R₁ ligands in the presence or in the absence of a base selected from the group consisting of K⁺(CH₃)₃CO—, CH₃NaO, K₂CO₃, triethylamine or N,N-diisopropylethylamine; and (ii) reacting the obtained compound, isolated or not isolated, with A-R₂ or BC-R₃ ligands in the presence or in the absence of a halide abstraction reagent selected from the group consisting of AgCF₃SO₃, AgBF₄, AgPFe, AgNO₂, AgNO₃, AgSO₄, NaCF₃SO₃, NaBF₄, NaPF₆, NaBArF, KCF₃SO₃, NH₄CF₃SO₃, or TlCF₃SO₃.

In process II, Cp is a cyclopentadienyl ring, L is a A-R₂ ligand or a monodentate ligand, X is a halide selected from the group consisting of fluoride, chloride, bromide, or iodide, n is an appropriate integer number to fulfill the metal coordination number preferably selected from 1 to 6 and Z is an alkali metal selected from the group consisting of Li, Na, or K.

Process III—directly reacting a compound of formula [MLnXn] or its solvates or its salts with Cp-R₁ or Z Cp-R₁ and A-R₂ or BC-R₃ ligands in the presence or in the absence of (a) a base selected from the group consisting of K⁺(CH₃)₃CO—, CH₃NaO, K₂CO₃, triethylamine or N,N-diisopropylethylamine; and (b) a halide abstraction reagent selected from the group consisting of AgCF₃SO₃, AgBF₄, AgPFe, AgNO₂, AgNO₃, AgSO₄, NaCF₃SO₃, NaBF₄, NaPF₆, NaBArF, KCF₃SO₃, NH₄CF₃SO₃, or TlCF₃SO₃.

In process III, Cp is a cyclopentadienyl ring, L is a A-R₂ ligand or a monodentate ligand, X is a halide selected from the group consisting of fluoride, chloride, bromide, or iodide; n is an appropriate integer number to fulfill the metal coordination number preferably selected from 1 to 6 and Z is an alkali metal selected from the group consisting of Li, Na, or K.

Process IV—comprises the steps of (i) reacting a compound of formula [M(Cp-R₁)L₃] or its solvates or its salts with one or two A-R₂ ligands or one BC-R₃ ligand; and (ii) reacting the obtained compound, isolated or not isolated, with A-R₂ or BC-R₃ ligands.

Process V—directly reacting a compound of formula [M(Cp-R₁)L₃] or its solvates or its salts with A-R₂ and BC-R₃ ligands.

In both processes IV and V, Cp is a cyclopentadienyl ring and L is a A-R₂ ligand or a monodentate ligand.

Process VI—comprises the steps of (i) reacting a metal salt of formula MXₙ or MWₙ or their solvates with CpR₁ or ZCpR₁, and A-R₂ or BC-R₃ ligands in the presence or in the absence of a base selected from the group consisting of K⁺(CH₃)₃CO—, CH₃NaO, K₂CO₃, triethylamine, or N,N-diisopropylethylamine; and (ii) reacting the obtained compound, isolated or not isolated, with A-R₂ or BC-R₃ ligands in the presence or in the absence of an abstraction reagent selected from the group consisting of AgCF₃SO₃, AgBF₄, AgPFe, AgNO₂, AgNO₃, AgSO₄, NaCF₃SO₃, NaBF₄, NaPF₆, NaBArF, KCF₃SO₃, NH₄CF₃SO₃, or TlCF₃SO₃.

In process VI, Cp is a cyclopentadienyl ring, X is a halide selected from the group consisting of fluoride, chloride, bromide, or iodide; W is an anion selected from the group consisting of sulfate, phosphate, nitrate, nitrite, acetate, citrate, perchlorate, carbonate, or oxide; n is an appropriate integer number to fulfill the metal coordination number preferably selected from 1 to 6 and Z is an alkali metal selected from the group consisting of Li, Na, or K.

Process VII—directly or stepwise reacting a metal salt of formula MXₙ or MWₙ or their solvates with CpR₁ or ZCpR₁ and A-R₂ and BC-R₃ ligands in the presence or in the absence of (a) a base selected from the group consisting of K⁺(CH₃)₃CO—, CH₃NaO, K₂CO₃, triethylamine, or N,N-diisopropylethylamine; and (b) a halide abstraction reagent selected from the group consisting of AgCF₃SO₃, AgBF₄, AgPFe, AgNO₂, AgNO₃, AgSO₄, NaCF₃SO₃, NaBF₄, NaPF₆, NaBArF, KCF₃SO₃, NH₄CF₃SO₃, or TlCF₃SO₃.

In process VII, Cp is a cyclopentadienyl ring, X is a halide selected from the group consisting of fluoride, chloride, bromide, or iodide; W is an anion selected from the group consisting of sulfate, phosphate, nitrate, nitrite, acetate, citrate, perchlorate, carbonate, or oxide; n is an appropriate integer number to fulfill the metal coordination number preferably selected from 1 to 6 and Z is an alkali metal selected from the group consisting of Li, Na, or K.

Process VIII—directly or stepwise reacting a dimeric compound of formula [M(Cp-R₁)X₂]₂ or its solvates or its salts with A-R₂ and/or BC-R₃ ligands and optionally isolating the intermediate compounds in the presence or in the absence of (a) a base selected from the group consisting of K⁺(CH₃)₃CO—, CH₃NaO, K₂CO₃, triethylamine, or N,N-diisopropylethylamine; and (b) a halide abstraction reagent selected from the group consisting of AgCF₃SO₃, AgBF₄, AgPFe, AgNO₂, AgNO₃, AgSO₄, NaCF₃SO₃, NaBF₄, NaPF₆, NaBArF, KCF₃SO₃, NH₄CF₃SO₃, or TlCF₃SO₃.

In process VIII, Cp is a cyclopentadienyl ring and X is a halide selected from the group consisting of fluoride, chloride, bromide, or iodide.

Process IX—directly or stepwise reacting a compound of formula [M(CpR₁)(Ar)] or its solvates or its salts with A-R₂ and/or BC-R₃ ligands and optionally isolating the intermediate compound.

In process IX, Cp is a cyclopentadienyl ring and Ar is an arene ligand selected from the group consisting of Cp-R₁, benzene, toluene, xylene, para-cymene, naphthalene, anthracene, phenanthrene, or indene.

Process X—reacting a compound of formula [M(Cp-F₁)(A-F₂)(BC-F₃)] or its solvates or its salts with a reactant or group of reactants to obtain the organometallic complex by addition, elimination, substitution, abstraction, conjugation, coupling, condensation, esterification, amidation, amination, alkylation, rearrangement, metathesis, cyclization, pericyclization, cycloaddition, hydrolysis, hydrogenation, redox, metalation, or any other suitable chemical reaction.

In process X, Cp is a cyclopentadienyl ring and F₁, F₂, and F₃ independently represent a chemical group which upon reaction, functionalization, or modification can be converted to R₁, R₂, and R₃, respectively.

For said processes of preparation (I to X), the reactions are carried out in any suitable solvent or mixture of solvents, preferably selected from the group consisting of water, acetone, toluene, hexane, methanol, tetrahydrofuran, pyridine, dichloromethane, dioxane, chloroform, ethanol, acetic acid, carbon tetrachloride, tert-butanol, dimethylglyoxime, trifluoroacetic acid, ethylene glycol, cyclohexane, dichloroethane, isopropanol, pentane, diethyl ether, propanol, dimethylformamide, benzene, dimethyl sulfoxide, ethyl acetate, xylene, butanol, dioxane, or heptane.

The temperature ranges from −20° C. to 150° C. and the pressure ranges from 0.1 and 10 atm; with or without stirring; under atmosphere of inert gas selected from dinitrogen or argon and in the presence or in the absence of irradiation by ultrasounds, microwaves, infrared light, ultraviolet light or visible light; and with or without the addition of a salt, acid, base, catalyst or coupling reagent.

It is also an object of the present invention a controlled-release multi-functional drug which comprises the organometallic complex as a self-dissociating portion thereof.

The controlled-release multi-functional drug of the present invention has the general Formula II

OC-L-TU (Formula II)
wherein
OC is the organometallic complex as defined in any of claims 1-4, or a pharmaceutically acceptable salt or solvate thereof,
TU is a targeting unit selected from the group consisting of peptide, protein, amino acid, peptidomimetic, antibody, affibody, nanobody, aptamer, antigen, or binding fragments thereof, which are able to selectively bind to receptors or drug targets overexpressed at cancer cell surfaces,
   wherein the targeting unit TU has a R₄ group in its structure, the R₄ group being selected from the group consisting of H, C₁-C₆ alkyl, C₁-C₆ aryl, —YCOY′, —YCHO, —YCONHNH₂, —YNHNH₂, —YCOOH, —YCOOY′, —YOCOOH, —YOCOOY′, —YNHCOOH, —YNHCOOY′, —YNHY′, —YONH₂, —YCH(CH₂)ₙ(OH)₂, —YPh(OH)₂, —YSH; —YCH(CH₂)ₙ(SH)₂, —YPh(SH)₂, —YB(OH)₂, —YOB(OH)₂, —YOPO₃H₂, —YOPO₂H₂, —Y(CH₂)ₙX, —YCOX, —YCHCH₂, —YN₃, —Y-imidazole, —YOCOO(CH₂)₂SH, —YNHCOO(CH₂)₂SH and —Y-AA,
      wherein
      Y and Y′ are independently selected from the group consisting of H, C₁-C₆ alkyl and C₁-C₆ aryl; each of said alkyl or aryl being unsubstituted or substituted by carbonyl, amide, ester, ether or thioether;
      n is an integer selected from 0 to 6;
      Ph is a phenyl group;
      X is a halogen atom selected from the group consisting of F, Cl, Br and I;
      the heteroaryl ring is a 5-12 membered heteroaryl ring wherein each heteroaryl ring optionally contains 1, 2 or 3 heteroatoms selected from the group consisting of oxygen, sulfur, phosphorus, nitrogen and selenium; and
      AA is a cis-aconitic anhydride group;
the organometallic complex OC and the targeting unit TU being bonded via a linking moiety L between the organometallic complex OC and the targeting unit TU;
   wherein the linking moiety L results from the reaction of the R₄ from the targeting unit TU with the R′ from the organometallic complex OC;
   wherein the R′ from organometallic complex OC is any one of R₁, R₂ or R₃;
   wherein at least one linking moiety L and at least one targeting unit TU are bonded in the same R′ or in different R′;
   wherein the linking moiety L is selected from the group consisting of —YC(Y′)═NNH—; —YNHN═C(Y′)—; —YC(Y′)═NNHC(O)—; —YC(O)NHN═C(Y′)—; —YC(Y′)═NC—; —C(Y′)═NCY—; —YPhC(Y′)═NC—; —YC(Y′)═NPh-; —YN═NY′—; —YC(O)NY′—; —C(O)N(Y′)Y—; cis-aconityl; —YC(Y′)═NO—; —C(Y′)═NOY—; —YC(Y′)(OY″)O—; —YC(Y′)(OH)O—; —YC(O)OC—; —YC(OY′)(OY″)OC—; —YOB(OY′)OC—; —YB(OY′)OC—; —YOC(O)OC—; bis-carbonate; para-aminobenzylcarbonate; —YOC(O)NY′—; —OC(O)N(Y′)Y—; para-methoxybenzyl carbamate, —YOC(O)CH₂CH₂SY′—; N-ethoxybenzylimidazole; N-alkoxyalkylimidazole; phosphoramidyl; phosphoramidate; —YSSY′—; disulphide carbamate, —YC(Y′)(SY″)S—; bis-alkylcarbonate dissulphide; bis-alkylcarbonate thioketal; —YSY′—; β-glucuronide; β-galactoside; or trityl,
      wherein
      Y, Y′ and Y″ are independently selected from the group consisting of H, C₁-C₆ alkyl and C₁-C₆ aryl; each of said alkyl or aryl being unsubstituted or substituted by carbonyl, amide, ester, ether or thioether;
      n is an integer selected from 0 to 6;
      Ph is a phenyl group; and
      the heteroaryl ring is a 5-12 membered heteroaryl ring wherein each heteroaryl ring optionally contains 1, 2 or 3 heteroatoms selected from the group consisting of oxygen, sulfur, phosphorus, nitrogen and selenium.

In a preferred embodiment of the present invention, the targeting unit TU is a peptide having a R₄ group in one of its end, the R₄ group being selected from the group consisting of H, CO(CH₂)₂COCH₃, CO(CH₂)₂CONHNH₂ and CO(CH₂)₂CO₂CO(CH₂)₂CONHNH₂.

In a preferred embodiment of the present invention, the linking moiety L has a hydrolytically cleavable bond and is selected from the group consisting of CONHNC(CH₃)(CH₂)₂CO, C(CH₃)NNHCO(CH₂)₂CO, C(CH₃)NNHCO(CH₂)₂CO, C(CH₂CH₃)NNHCO(CH₂)₂CO and C(CH₂Ph)NNHCO(CH₂)₂CO.

In the most preferred embodiment of the invention, the controlled-release multi-functional drug is

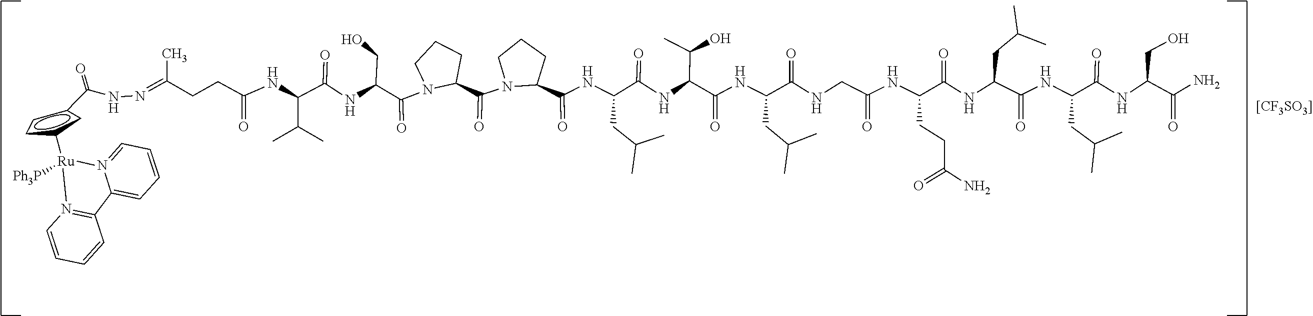

or (2,2′-bipyridyl)(triphenylphosphane)(4-((E,Z)-2-η⁵-cyclopentadienyl formylhydrazineylidene)pentanoyl-VSPPLTLGQLLS)ruthenium(II) trifluoromethanesulfonate;

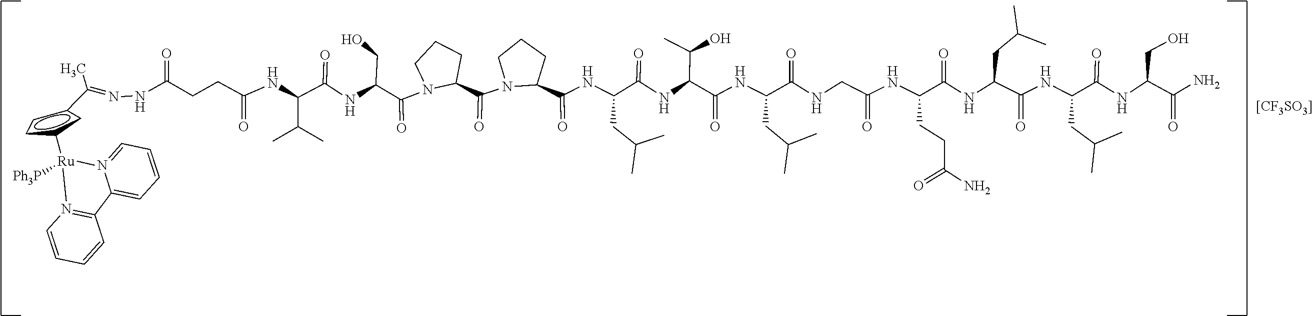

or (2,2′-bipyridyl)(triphenylphosphane)(4-((E,Z)-2-(1-η⁵-cyclopentadienyl ethylidene)hydrazineyl)-4-oxobutanoyl-VSPPLTLGQLLS)ruthenium(II) trifluoromethanesulfonate; and

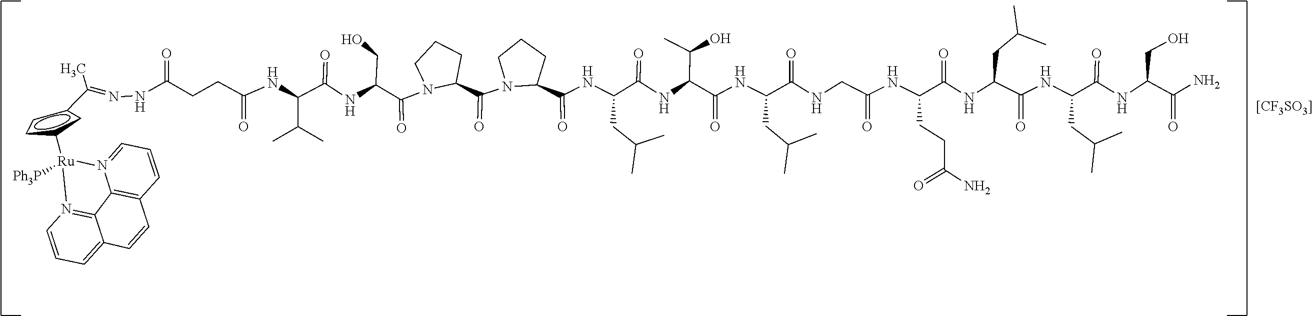

or (1,10-phenanthroline)(triphenylphosphane)(4-((E,Z)-2-(1-η⁵-cyclopentadienyl ethylidene)hydrazineyl)-4-oxobutanoyl-VSPPLTLGQLLS)ruthenium(II) trifluoromethanesulfonate.

The controlled-release multi-functional drugs of formula II can be cationic or neutral and can be in salt form, whose anions are selected from the group consisting of halides, phosphates, triflates, phenylborates and hexafluorophosphates.

The controlled-release multi-functional drugs of the present invention are air stable and stable in aqueous media for an appropriate period for medicinal purposes and have controlled release profile favorable to the selective delivery of the active organometallic complexes into the tumor/metastases and very relevant antitumor properties.

The present invention further provides, in another aspect, processes for the preparation of the controlled-release multi-functional drugs of formula II as described above, or a pharmaceutically acceptable salt and/or solvate thereof.

In a preferred embodiment of the application, the processes for the preparation of the controlled-release multi-functional comprise the step of reacting the organometallic complex with one or more targeting units containing a R₄ group, wherein the R₁, R₂ and/or R₃ groups of the organometallic complex is reacted with the R₄ by means of conjugation, coupling, cross-coupling, condensation, rearrangement, addition, elimination, substitution, cycloaddition, redox, or any other suitable chemical reaction in a solvent or a mixture of solvents that allow and/or facilitate said reaction, in the presence or the absence of a catalyst or coupling agent.

In a preferred embodiment of the present invention, the solvent or mixture of solvents is selected from the group consisting of water, acetone, toluene, hexane, methanol, tetrahydrofuran, pyridine, dichloromethane, dioxane, chloroform, ethanol, acetic acid, carbon tetrachloride, tert-butanol, dimethylglyoxime, trifluoroacetic acid, ethylene glycol, cyclohexane, dichloroethane, isopropanol, pentane, diethyl ether, propanol, dimethylformamide, benzene, dimethyl sulfoxide, ethyl acetate, xylene, butanol, dioxane, or heptane.

In a preferred embodiment of the present invention, the temperature ranges from −20° C. to 150° C. and the pressure ranges from 0.1 and 10 atm; with or without stirring; under atmosphere of inert gas selected from dinitrogen or argon and in the presence or in the absence of irradiation by ultrasounds, microwaves, infrared light, ultraviolet light or visible light; and with or without the addition of a salt, acid, base, catalyst or coupling reagent.

Also, the products are obtained without further purification or isolated and further purified by any suitable analytical method selected from the group consisting of crystallization, recrystallization, co-crystallization, sublimation, distillation, chromatography, filtration, precipitation, or extraction.

In a further aspect of the present invention, the organometallic complexes or a pharmaceutically acceptable salt or solvate thereof and the controlled-release multi-functional drugs or a pharmaceutically acceptable salt or solvate thereof are used, alone or in combination with an approved drug and/or pharmaceutical product, as an antitumoral agent and/or anti-metastatic agent and/or as a radio sensitizer and/or diagnosis agent and/or theragnostic agent in cancer therapy, prevention and/or identification, independently of the stage of the disease.

The cancer is either primary cancers and/or cancer metastasis, selected from the group consisting of breast cancer, lymphoma, skin cancer, ovarian cancer, liver cancer, cervix cancer, head and neck cancer, colon cancer, testicular cancer, larynx cancer, nasopharynx cancer, bladder cancer, oropharynx cancer, lung cancer, rectal cancer, brain cancer, prostate cancer, stomach cancer, kidney cancer, esophageal cancer, thyroid cancer, pancreatic cancer, bone cancer, or leukemia.

In a preferred embodiment of the present application, the organometallic complexes or a pharmacologically acceptable salt and/or solvate thereof and the controlled-release multi-functional drugs or a pharmaceutically acceptable salt or solvate thereof is administered to a subject in need via topical, intravenous, subcutaneous or intraperitoneal administration.

It is also an object of the present invention a pharmaceutical composition comprising a pharmaceutically effective amount of an organometallic complex or a pharmacologically acceptable salt and/or solvate thereof or a pharmaceutically effective amount of a controlled-release multi-functional drug or a pharmaceutically acceptable salt or solvate thereof and at least one active ingredient and/or pharmaceutically acceptable carrier, excipient, or diluent.

In a preferred embodiment of the invention, the pharmaceutical composition is for use, alone or in combination with an approved drug and/or pharmaceutical product, as an antitumoral agent and/or anti-metastatic agents and/or as a radio sensitizer agent in cancer therapy and/or cancer prevention, independently of the stage of the disease.

The cancer is either primary cancers and/or cancer metastasis, selected from the group consisting of breast cancer, lymphoma, skin cancer, ovarian cancer, liver cancer, cervix cancer, head and neck cancer, colon cancer, testicular cancer, larynx cancer, nasopharynx cancer, bladder cancer, oropharynx cancer, lung cancer, rectal cancer, brain cancer, prostate cancer, stomach cancer, kidney cancer, esophageal cancer, thyroid cancer, pancreatic cancer, bone cancer, or leukemia.

In a preferred embodiment of the present application, the pharmaceutical composition is administered to a subject in need via topical, intravenous, subcutaneous or intraperitoneal administration.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular.

Generally, nomenclatures used in connection with, and techniques of synthesis of compounds described herein are those well-known and commonly used in the art. Known methods, processes and techniques are generally performed according to conventional methods well known in the art and as described in various general and more specific references that are discussed throughout the present specification, unless otherwise indicated. Also, the nomenclatures used in connection with laboratory procedures and techniques described herein are those well-known and commonly used in the art.

The following terms, unless otherwise indicated, shall be understood to have the following meanings:

The term “multi-functional drug” refers to a system having an organometallic complex, a linking moiety and a targeting unit.

The term “monodentate ligand” refer to any synthetic or natural molecule that possess one atom with electronic availability to form coordination bonds to the transition metal. The monodentate ligands are preferably selected from the group consisting of heteroaromatic ligands, phosphanes, —CO, —NO, nitriles, isonitriles, a metal complex or a molecule of biological interest.

The term “bidentate ligand” refers to any synthetic or natural molecule having two atoms with electronic availability to form coordination bonds to the same transition metal. The bidentate ligands are preferably selected from the group consisting of heteroaromatic ligands, diphosphanes, a metal complex or a bidentate molecule of biological interest.

The term “heteroaromatic ligand” refers to any molecule with a single ring or multiple ring system in which at least one of the rings has at least one heteroatom, wherein the heteroatoms in the heteroaromatic ligands are the same heteroatoms or different heteroatoms, the heteroatoms being independently selected from group consisting of O, S, P, N or Se.

The term “biomolecule” refers to any chemical compound existing in living beings or a molecule that mimics a biomolecule, synthesized by chemical processes, having biological properties. In the context of the present invention, preferred biomolecules are, but without limitation, an amino acid, a peptide, a protein, a protein fragment, a peptidomimetic, an aptamer, an antibody, an affibody, a nanobody, an antigen, a binding fragment, a vitamin, a carbohydrate, a nucleic acid, among others. Some biomolecules due to its chemical structure that contains heteroaromatic atoms can themselves be recognized as a “heteroaromatic ligand”.

The term “molecule of biological interest” refers to any natural or synthetic molecule with potential bioactivity. A compound is considered “bioactive” if it has an interaction or effect towards any cellular living tissue.

The term “linking moiety” refers to any chemical function sensitive to tumor environment stimuli that triggers the controlled release of the active organometallic complex. Preferably, the linking moiety is selected from the group consisting of YC(Y′)═NNH—; —YNHN═C(Y′)—; —YC(Y′)═NNHC(O)—; —YC(O)NHN═C(Y′)—; —YC(Y′)═NC—; —C(Y′)═NCY—; —YPhC(Y′)═NC—; —YC(Y′)═NPh-; —YN═NY′—; —YC(O)NY′—; —C(O)N(Y′)Y—; cis-aconityl; —YC(Y′)═NO—; —C(Y′)═NOY—; —YC(Y′)(OY″)O—; —YC(Y′)(OH)O—; —YC(O)OC—; —YC(OY′)(OY″)OC—; —YOB(OY′)OC—; —YB(OY′)OC—; —YOC(O)OC—; bis-carbonate; para-aminobenzylcarbonate; —YOC(O)NY′—; —OC(O)N(Y′)Y—; para-methoxybenzyl carbamate, —YOC(O)CH₂CH₂SY′—; N-ethoxybenzylimidazole; N-alkoxyalkylimidazole; phosphoramidyl; phosphoramidate; —YSSY′—; disulphide carbamate, —YC(Y′)(SY″)S—; bis-alkylcarbonate dissulphide; bis-alkylcarbonate thioketal; —YSY′—; pi-glucuronide; pi-galactoside; or trityl.

By “targeting unit”, it is meant any synthetic or natural entity with high affinity and selectivity to bind the receptors or drug targets overexpressed at the cancer cell surfaces; preferred target unit is, but without limitation, a peptide, an amino acid, a peptidomimetic, an antibody, an affibody, an nanobody, a protein, an aptamer, an antigen, a pharmaceutical composition, its analogues, derivatives, or binding fragments and or any molecule or biomolecule with or that keeps the affinity for the receptors.

For the purpose of the present invention, the terms “receptors and drug targets” refers to any protein, glycoprotein, lipoprotein, enzyme, other biomacromolecules (which contain amino acids, glucosides, lipids and/or nucleic acids), membrane channels, membrane transport proteins, cell organelles or their fragment that are known targets of drugs and/or can be recognized by the targeting units above mentioned and whose expression, structure and/or activity are altered in tumor tissues/cancer cells (e.g. overexpression, overactivation, among others).

By “tumor environment stimuli” and “tumor microenvironment stimuli” are meant any property characteristic of the tumor tissues or its environment; preferred tumor environment stimuli are, but without limitation, pH value; glutathione (GSH/GSSH) level; reactive oxygen species levels (e.g., H₂O₂, O₂·, ·OH, ONOO—, OCl—, superoxide, among others); adenosine triphosphate (ATP) level; oxygen level (e.g. hypoxia); redox stimuli; inflammatory mediators levels (e.g. tumour necrosis factor, interleukins, cytokines, immune cells, among others); and enzymes whose expression and/or enzymatic activity is higher or alternated or specific of tumour tissues/cancer cells (e.g. cathepsin B, cathepsin D, cathepsin L, cathepsin K, legumain, hyaluronidases, matrix metalloproteinases, esterases, amidases, amylases, proteases, azoreductases, nitroreductases, cytochromes, glycosidases, β-galactosidase, β-glucuronidase, among others).

By “metal salts” (MXₙ or MWₙ) are meant halide, nitrate, fluoride, chloride, bromide, iodide, phosphate, perchlorate, or carbonate salts represented by X, or sulfate, phosphate, nitrate, nitrite, acetate, citrate, perchlorate, carbonate, oxide anions represented by W, of any suitable transition metal represented by M, in particular ruthenium, iron, rhodium, cobalt, iridium, osmium, molybdenum, manganese, rhenium, or technetium.

For the purpose of the present invention, the term “pharmaceutically acceptable” is meant what is useful to the preparation of a pharmaceutical composition, and what is generally safe and non-toxic, for a pharmaceutical use.

By “pharmaceutically acceptable salt and/or solvate” is meant a salt and/or solvate of a compound which is pharmaceutically acceptable, as defined above, and which holds the pharmacological activity of the corresponding compound.

For the purpose of the present invention, by “pharmaceutical composition” is meant a composition having diagnosis and/or preventive and/or curative properties towards cancers.

The particular examples presented below are intended only to illustrate the present invention and should not be construed as limitation of the present invention.

### EXAMPLES

### Synthesis and Characterization

### A) Materials

All chemicals were obtained from standard suppliers and used without further purification. The synthesis of the organometallic complexes and the multi-functional drugs were carried out under dinitrogen atmosphere using air-free Schlenk techniques and dry solvents.

Organic solvents were dried before use by passage through a solvent purification system MBraun SPS-800 (4.8 L, filter material MB-KOL-A/MB-KOL-M, catalyst MB-KOL-C) under dinitrogen atmosphere. Water was demineralized before use by passage through a Millipore Milli-Q water purification system.

The peptides were prepared by fluorenylmethyloxycarbonyl (fmoc)-based ultrasound-assisted solid-phase peptide synthesis methods in a polypropylene syringe reactor with incorporated filter frit and removable cap (5 mL, Multisyntech GmbH). Ultrasonication was performed in a water bath (240×137×100 mm, Elmasonic Elma S30H) at 37 kHz and 30° C.

The starting materials chlorobis(triphenylphosphane)(η⁵-cyclopentadienyl) ruthenium(II) ([RuCp(PPh₃)₂Cl]), (2,2′-bipyridyl)(triphenylphosphane)(η⁵-ethyl cyclopentadienylcarboxylate)ruthenium(II) trifluoromethanesulfonate ([Ru(CpCO₂CH₂CH₃)(PPh₃)(bipy)][CF₃SO₃]), and 4-hydroxy-4′-methoxy-2,2′-bipyridine (bipy-OH) were synthesized as previously reported (Bruce, M. I. et. al, 1977; Franco Machado, J. et. al, 2020; Sand, H. et. al, 2015).

### B) Instruments and Methods

Nuclear magnetic resonance (NMR) spectra were collected on a Bruker Avance 400 spectrometer at probe temperature (1H NMR at 400 MHz, 13C NMR at 100.6 MHz, and 31P NMR at 161.9 MHz) using deuterated solvents.

Fourier-transform infrared (FT-IR) spectra (4000-250 cm⁻¹) were recorded in KBr at room temperature on a Thermo Nicolet 6700 spectrophotometer.

Ultraviolet-visible (UV-Vis) spectra (235-900 nm) were acquired at room temperature on a Jasco V-660 spectrometer, using quartz cuvettes (1 cm optical path length) for solutions of the compounds at concentrations ranging from 1×10⁻⁵ M to 1×10⁻³ M.

Elemental analyses (EA) were performed on a Fisons Instruments EA1108 system at the Laboratório de Anelises of Instituto Superior Técnico (University of Lisbon).

Electrospray ionization mass spectrometry (ESI-MS) spectra (100-3000 m/z) were collected at room temperature on a QITMS instrument (Bruker HCT) in positive ionization mode, using acetonitrile as solvent.

Semi-preparative reversed-phase high performance liquid chromatography (RP-HPLC) analyses were performed on a Waters 2535 instrument coupled to a Uniflows DG-3210 degasser and a Waters 2998 UV-Vis detector, using either a C18 reversed-phase column (8×250 mm, 7 μm, Macherey-Nagel VP250/8 Nucleosil 100-7) coupled to a pre-column (8×30 mm, 7 μm, Macherey-Nagel VP30/8 Nucleosil 100-7) for methods 1 and 2, or a C18(2) reversed-phase column (10×150 mm, 10 μm, Phenomenex Luna 00F-4253-N0) coupled to a pre-column (10×10 mm, Phenomenex Security Guard AJ0-7221) for method 3. The eluents used were 0.1% (v/v) trifluoroacetic acid (TFA) in water (mobile phase A) and 0.1% (v/v) TFA in acetonitrile (mobile phase B) for methods 1 and 2, or 10 mM NH4HCO3 in water (pH=8, mobile phase C) and acetonitrile (mobile phase D) for method 3. Applied binary gradients were 0→5 min: 10% B; 5→35 min: 10%→100% B; 35→38 min: 100% B; 38→40 min: 100%→10% B; 40→45 min: 10% B at 2 mL·min⁻¹ flow rate (method 1), or 0→5 min: 10% B; 5-30 min: 10%→40% B; 30→45 min: 40% B; 45→50 min: 40→100% B; 50→53 min: 100% B, 53→55 min: 100→10% B, 55→60 min: 10% B at 2 mL·min⁻¹ flow rate (method 2), or 0→3 min: 10% D; 3→5 min: 10%→70% D; 5→10 min: 70% D; 10→20 min: 70%→90% D; 20→25 min: 90% D; 25→27 min: 90%→10% D; 27→30 min: 10% D at 3 mL·min⁻¹ flow rate (method 3). The samples were dissolved in 9:1 (v/v) A:B (methods 1 and 2) or 9:1 (v/v) C:D (method 3) and filtered through a 0.45 μm membrane filter prior to injection. Detection was performed by UV-Vis at 200-600 nm. Analytical RP-HPLC analyses were performed on a PerkinElmer Series 200 instrument coupled to a PerkinElmer Series 200 degasser and a PerkinElmer Series 200 UV-Vis detector, using either a C18 reversed-phase column (4.6×250 mm, 5 μm, Supelco Analytical 568223-U) coupled to a pre-column (4.0×20 mm, 5 μm, Supelco 568273-U Discovery) for method 4, or a C18(2) reversed-phase column (4.6×150 mm, 3 μm, Phenomenex Luna 00F-4251-E0) coupled to a pre-column (3.0×4 mm, Phenomenex Security Guard AJ0-928) for method 5. The eluents used were mobile phase A and mobile phase B for method 4, or mobile phase C and mobile phase D for method 5. Applied binary gradients were 0→5 min: 10% B; 5→20 min: 10%→100% B; 20→25 min: 100% B; 25→27 min: 100%→10% B; 27-30 min: 10% B at 1 mL·min⁻¹ flow rate (method 4), or 0→3 min: 10% D; 3→5 min: 10%→70% D; 5→10 min: 70% D; 10→20 min: 70%→90% D; 20→25 min: 90% D; 25→27 min: 90%→10% D; 27→30 min: 10% D at 0.5 mL·min⁻¹ flow rate (method 5). The samples were dissolved in 9:1 (v/v) A:B (method 4) or 9:1 (v/v) C:D (method 5) and filtered through a 0.45 μm membrane filter prior to injection. Detection was performed by UV at 220 nm.

### C) Synthesis of Organometallic Complexes (Ruthenium Complexes)

### Example 1. Synthesis of chlorobis(triphenylphosphane)(η⁵-methyl cyclopentadienylketone)ruthenium(II) ([Ru(CpCOCH₃)(PPh)₂Cl]), complex 1

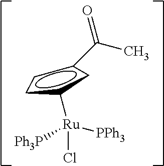

First, freshly cracked cyclopentadiene (2.5 mL, 30 mmol) was added to a stirring slurry of sodium sand (0.17 g, 7.5 mmol) in tetrahydrofuran (10 mL) at 0° C. After all the sodium has reacted, methyl acetate (2.4 mL, 30 mmol) was added to the resulting slightly pink solution of sodium cyclopentadienide. The mixture was refluxed for 2 h. Then, the solvent was removed under vacuum. The resulting purple-brown solid was washed with diethyl ether (3×15 mL) and vacuum dried. Sodium acetylcyclopentadienide was afforded as a white powder. Yield: 74%.

Afterwards, dichlorotris(triphenylphosphane)ruthenium(II) (3.2 g, 3.3 mmol) was added to a stirring solution of sodium acetylcyclopentadienide (0.55 g, 4.2 mmol) in tetrahydrofuran (30 mL). The mixture acquired black color and was stirred overnight at room temperature. The resulting brick-red precipitate was cannula filtered, washed with cold tetrahydrofuran (3×20 mL) and vacuum dried. The product was then recrystallized from dichloromethane/n-hexane, affording burgundy-red crystals. Yield: 70%.

¹H NMR [CDCl₃, Me₄Si, δ/ppm]: 7.36 (m, 12H), 7.23 (t, 6H, ³J=6.73 Hz), 7.12 (m, 12H), 5.12 (br, 2H), 3.62 (br, 2H), 2.21 (s, 3H). ¹³C NMR [CDCl₃, δ/ppm]: 197.18, 137.31 (t, ¹J=21.04 Hz), 134.00 (t, ²J=4.86 Hz), 129.11, 127.61 (t, ³J=4.60 Hz), 88.13, 86.71, 79.08, 29.57. ³¹P NMR [CDCl₃, δ/ppm]: 37.36 (s). FT-IR [KBr, cm^{-1]: 3101}-3047 (vC-H, aromatic), 2858 (vC-H, aliphatic), 1680 (vC═O), 1481-1354 (vC═C, aromatic+δC—H, aliphatic), 1273-692 (δC—H, aromatic). UV-Vis [DCM, λmax/nm (ε/M⁻¹·cm⁻¹)]: 290 (sh), 394 (2.70×10³). EA (%) found: C 66.6, H 4.8. Calculated for C₄₃H₃₇RuClOP₂·0.1CH₂Cl₂ (776.71 g/mol): C 66.7, H 4.8.

### Example 2. Synthesis of chlorobis(triphenylphosphane)(η⁵-ethyl cyclopentadienylketone)ruthenium(II) ([Ru(CpCOCH₂CH₃)(PPh₃)₂Cl]), complex 2

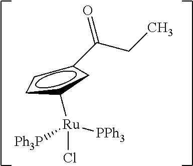

First, freshly cracked cyclopentadiene (2.5 mL, 30 mmol) was added to a stirring slurry of sodium sand (0.17 g, 7.5 mmol) in tetrahydrofuran (10 mL) at 0° C. After all the sodium has reacted, methyl propionate (2.9 mL, 30 mmol) was added to the resulting slightly pink solution of sodium cyclopentadienide. The mixture was refluxed for 2 h. Then, the solvent was removed under vacuum. The resulting purple-brown solid was washed with diethyl ether (3×15 mL) and vacuum dried. Sodium propionylcyclopentadienide was afforded as a white powder. Yield: 52%.

Afterwards, dichlorotris(triphenylphosphane)ruthenium(II) (1.5 g, 1.65 mmol) was added to a stirring solution of sodium propionylcyclopentadienide (0.3 g, 2.1 mmol) in tetrahydrofuran (30 mL). The mixture acquired black color and was stirred overnight at room temperature. The resulting brick-red precipitate was cannula filtered, washed with cold tetrahydrofuran (3×20 mL) and vacuum dried. The product was then recrystallized from dichloromethane/n-hexane, affording burgundy-red crystals. Yield: 79%.

¹H NMR [CDCl₃, Me₄Si, δ/ppm]: 7.34 (m, 12H), 7.23 (m, 6H), 7.13 (m, 12H), 5.06 (br, 2H), 3.57 (br, 2H), 2.65 (br, 2H), 1.06 (br, 3H). ¹³C NMR [CDCl₃, δ/ppm]: 199.70, 137.50 (t, ¹J=20.90 Hz), 133.98 (t, ²J=4.80 Hz), 129.07, 127.64 (t, ³J=4.53 Hz), 88.88, 86.22, 79.01, 34.82, 7.65. ³¹P NMR [CDCl₃, δ/ppm]: 37.20 (s). FT-IR [KBr, cm^{-1]: 3081}-3047 (vC—H, aromatic), 2969-2867 (vC—H, aliphatic), 1672 (vC═O), 1481-1369 (vC═C, aromatic+δC—H, aliphatic), 1243-694 (δC—H, aromatic). UV-Vis [DCM, λmax/nm (ε/M⁻¹·cm⁻¹)]: 290 (sh), 393 (1.71×10³). EA (%) found: C 67.5, H 5.0. Calculated for C₄₄H₃₉RuClOP₂ (782.26 g/mol): C 67.6, H 5.0.

### Example 3. Synthesis of chlorobis(triphenylphosphane)(η⁵-phenylmethyl cyclopentadienylketone)ruthenium(i) ([Ru(CpCOCH₂Ph)(PPh₃)₂Cl]), complex 3

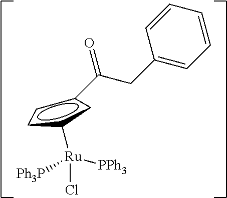

First, freshly cracked cyclopentadiene (2.5 mL, 30 mmol) was added to a stirring slurry of sodium sand (0.17 g, 7.5 mmol) in tetrahydrofuran (10 mL) at 0° C. After all the sodium has reacted, ethyl phenylacetate (4.8 mL, 30 mmol) was added to the resulting slightly pink solution of sodium cyclopentadienide. The mixture was refluxed for 2 h. Then, the solvent was removed under vacuum. The resulting purple-brown solid was washed with a 1:9 mixture of tetrahydrofuran/diethyl ether (3×15 mL) and vacuum dried. Sodium phenylacetylcyclopentadienide was afforded as a white powder. Yield: 59%.

Afterwards, dichlorotris(triphenylphosphane)ruthenium(II) (1.4 g, 1.5 mmol) was added to a stirring solution of sodium phenylacetylcyclopentadienide (0.35 g, 1.9 mmol) in tetrahydrofuran (30 mL). The mixture acquired black color and was stirred overnight at room temperature. The resulting orange precipitate was cannula filtered, washed with diethyl ether (3×20 mL) and vacuum dried. The product was then recrystallized from dichloromethane/n-hexane, affording orange crystals. Yield: 94%.

¹H NMR [CDCl₃, Me₄Si, δ/ppm]: 7.34 (m, 15H), 7.23 (m, 8H), 7.11 (m, 12H), 4.99 (br, 2H), 4.01 (s, 2H), 3.56 (br, 2H). ¹³C NMR [CDCl₃, δ/ppm]: 197.00, 137.52 (t, ¹J=21.08 Hz), 135.47, 133.99 (t, ²J_{PC}=4.90 Hz), 130.32, 129.14, 128.44, 127.70 (t, ³J=4.61 Hz), 126.53, 89.30, 85.97, 79.50, 48.61. ³¹P NMR [CDCl₃, δ/ppm]: 36.80 (s). FT-IR [KBr, cm⁻¹]: 3077-3056 (vC—H, aromatic), 2852 (vC—H, aliphatic), 1679 (vC═O), 1479-1378 (vC═C, aromatic+δC—H, aliphatic), 1243-696 (δC—H, aromatic). UV-Vis [DCM, λmax/nm (ε/M⁻¹·cm⁻¹)]: 290 (sh), 399 (2.14×10³). EA (%) found: C 69.1, H 5.0. Calculated for C₄₉H₄₁RuClOP₂·0.1CH₂Cl₂ (852.81 g/mol): C 69.1, H 4.9.

### Example 4. Synthesis of (2,2′-bipyridyl)(triphenylphosphane)(η⁵-cyclopentadienylhydrazide)ruthenium(1) trifluoromethanesulfonate ([Ru(CpCONHNH₂)(PPh₃)(bipy)][CF₃SO₃]), complex 4

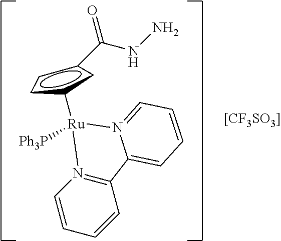

A solution of hydrazine hydrate in water (80% v/v, 1.8 mL, 37.5 mmol) was added to a stirring solution of [Ru(CpCO₂CH₂CH₃)(PPh₃)(bipy)][CF₃SO₃](0.20 g, 0.25 mmol) in ethanol (10 mL). The mixture was refluxed for 5 h, turning from orange to red. Then, the solvent was removed under vacuum. The resulting residue was washed with diethyl ether (2×10 mL) and vacuum dried. The product was then recrystallized from methanol/diethyl ether, affording orange crystals. Yield: 92%.

¹H NMR [(CD₃)₂SO, Me₄Si, δ/ppm]: 9.28 (d, 2H, ³J=4.37 Hz), 9.18 (br, 1H), 8.18 (d, 2H, ³J=7.77 Hz), 7.87 (t, 2H, ³J=7.06 Hz), 7.37 (m, 5H), 7.30 (m, 6H), 6.91 (t, 6H, 3J=8.24 Hz), 5.49 (br, 2H), 4.77 (br, 2H), 4.12 (br, 2H). ¹³C NMR [(CD₃)₂SO, 6/ppm]: 164.33, 155.66, 155.23, 136.62, 132.57 (d, ²J=10.95 Hz), 130.69 (d, ¹J=42.27 Hz), 130.18 (br), 128.51 (d, ³J=9.58 Hz), 125.24, 123.37, 82.21, 81.27, 77.09. ³¹P NMR [(CD₃)₂SO, δ/ppm]: 50.42 (s). FT-IR [KBr, cm⁻¹]: 3318 (vN—H), 3102-3058 (vC—H, aromatic), 1641 (vC═O), 1610-1608 (5N—H), 1479-1382 (vC═N+vC═C, aromatic), 1251 (vCF₃SO₃), 1226-698 (δC—H, aromatic). UV-Vis [DCM, λmax/nm (ε/M⁻¹·cm⁻¹)]: 289 (2.65×10⁴), 345 (sh), 409 (5.34×10³), 455 (sh). EA (%) found: C 53.2, H 3.9, N 6.9, S 4.0. Calculated for C₃₅H₃₀RuF₃N₄O₄PS (791.75 g/mol): C 53.1, H 3.8, N 7.1, S 4.0. RP-HPLC (method 5): t_{R}=26.1 min. ESI-MS (positive mode, m/z) found: 643.2 [M]+. Calculated for C₃₄H₃₀RuN₄OP (643.12 u): 643.1 [M]+.

### Example 5. Synthesis of (2,2′-bipyridyl)(triphenylphosphane)(η⁵-methyl cyclopentadienylketone)ruthenium(II) trifluoromethanesulfonate ([Ru(CpCOCH₃)(PPh₃)(bipy)][CF₃SO₃]), complex 5

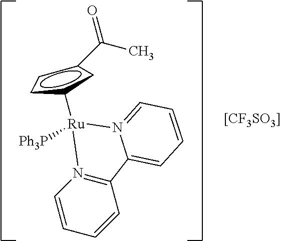

Silver trifluoromethanesulfonate (0.13 g, 0.5 mmol) and 2,2′-bipyridine (0.08 g, 0.5 mmol) were added to a stirring solution of [Ru(CpCOCH₃)(PPh₃)₂Cl] (complex 1, 0.38 g, 0.5 mmol) in methanol (20 mL). The mixture was refluxed for 4 h in dark, turning from brick-red to orange-red. Then, the solution was separated from the AgCl precipitate by cannula filtration and the solvent was removed under vacuum. The resulting residue was washed with n-hexane (2×10 mL), diethyl ether (2×10 mL), and vacuum dried. The product was then recrystallized from dichloromethane/diethyl ether, affording orange crystals. Yield: 68%.

¹H NMR [(CD₃)₂CO, Me₄Si, δ/ppm]: 9.42 (d, 2H), 8.23 (d, 2H, ³J=7.60 Hz), 7.98 (m, 2H), 7.45 (m, 5H), 7.35 (m, 6H), 7.12 (m, 6H), 5.78 (br, 2H), 4.70 (br, 2H), 1.66 (s, 3H). ¹³C NMR [(CD₃)₂CO, δ/ppm]: 195.36, 157.11, 156.55, 137.99, 133.89 (d, ²J=10.81 Hz), 131.86 (d, ¹J=42.37 Hz), 131.33 (br), 129.51 (d, ³J=9.80 Hz), 126.74, 124.45, 86.64, 84.78, 77.84, 27.11. ³¹P NMR [(CD₃)₂CO, δ/ppm]: 49.67 (s). FT-IR [KBr, cm^{-1]: 3110}-2998 (vC—H, aromatic), 2921 (vC—H, aliphatic), 1660 (vC═O), 1479-1336 (vC═N+vC═C, aromatic+δC—H, aliphatic), 1259 (vCF₃SO₃), 1222-700 (C—H, aromatic). UV-Vis [DCM, λmax/nm (ε/M⁻¹·cm⁻¹)]: 288 (1.86×10⁴), 350 (sh), 404 (3.62×10³), 450 (sh). EA (%) found: C 55.7, H 3.5, N 3.5, S 4.0. Calculated for C₃₆H₃₀RuF₃N₂O₄PS (775.74 g/mol): C 55.7, H 3.9, N 3.6, S 4.1. RP-HPLC (method 5): t_{R}=25.2 min. ESI-MS (positive mode, m/z) found: 627.3 [M]+. Calculated for C₃₅H₃₀RuN₂OP (627.11 u): 627.1 [M]+.

### Example 6. Synthesis of (1,10-phenanthroline)(triphenylphosphane)(η⁵-methyl cyclopentadienylketone)ruthenium(II) trifluoromethanesulfonate ([Ru(CpCOCH₃)(PPh₃)(phen)][CF₃SO₃]), complex 6

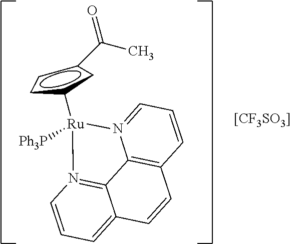

Silver trifluoromethanesulfonate (0.13 g, 0.5 mmol) and 1,10-phenanthroline (0.09 g, 0.5 mmol) were added to a stirring solution of [Ru(CpCOCH₃)(PPh₃)₂Cl](complex 1, 0.38 g, 0.5 mmol) in methanol (20 mL). The mixture was refluxed for 4 h in dark, turning from brick-red to brown-red. Then, the solution was separated from the AgCl precipitate by cannula filtration and the solvent was removed under vacuum. The resulting residue was washed with diethyl ether (2×10 mL) and vacuum dried. The product was then recrystallized from dichloromethane/diethyl ether, affording orange crystals. Yield: 36%.

¹H NMR [(CD₃)₂CO, Me₄Si, δ/ppm]: 9.79 (d, 2H, ³J=5.29 Hz), 8.55 (d, 2H, ³J=8.15 Hz), 8.03 (s, 2H), 7.84 (dd, 2H, ³J=8.2, 5.3 Hz), 7.31 (m, 3H), 7.19 (td, 6H, ³J=7.66 and 2.17 Hz), 6.99 (m, 6H), 5.92 (m, 2H), 4.78 (m, 2H), 1.53 (s, 3H). ¹³C NMR [(CD₃)₂CO, δ/ppm]: 195.15 (d), 157.46 (d, ¹J=1.89 Hz), 147.98, 136.88, 133.64 (d, ²J=10.88 Hz), 131.66, 131.47, 131.20 (d, ⁴J=2.23 Hz), 129.20 (d, ³J=9.86 Hz), 128.34, 126.02, 86.40 (d), 84.35 (d), 77.64, 27.12. ³¹P NMR [(CD₃)₂CO, δ/ppm]: 50.25 (s). FT-IR [KBr, cm^{-1]: 3080}-3050 (vC—H, aromatic), 1658 (vC═O), 1448 1429 (vC═C, aromatic), 1385 (vC═N, aromatic), 1355 (C—H, aliphatic), 1265 (vCF₃SO₃). UV-Vis [DCM, λmax/nm (ε/M⁻¹·cm⁻¹)]: 253 (3.79×10⁴), 378 (9.33×10³). EA (%) found: C 57.0, H 3.6, N 3.5, S 4.0. Calculated for C₃₈H₃₀RuF₃N₂O₄PS (799.76 g/mol): C 57.1, H 3.8, N 3.5, S 4.0. RP-HPLC (method 5): t_{R}=27.0 min. ESI-MS (positive mode, m/z) found: 651.3 [M]+. Calculated for C₃₇H₃₀RuN₂OP (651.11 u): 651.1 [M]+.

### Example 7. Synthesis of (pyrazino[2,3-f][1,10]phenanthroline) (triphenylphosphane)(η⁵-methyl cyclopentadienylketone)ruthenium(II) trifluoromethanesulfonate ([Ru(CpCOCH₃)(PPh₃)(pyzphen)][CF₃SO₃]), complex 7

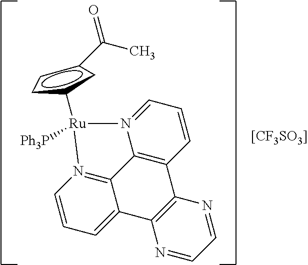

Silver trifluoromethanesulfonate (0.13 g, 0.5 mmol) and pyrazino[2,3-f][1,10]phenanthroline (0.12 g, 0.5 mmol) were added to a stirring solution of [Ru(CpCOCH₃)(PPh₃)₂Cl] (complex 1, 0.38 g, 0.5 mmol) in methanol (20 mL). The mixture was refluxed for 4 h in dark, turning from brick-red to brown-red. Then, the solution was separated from the AgCl precipitate by cannula filtration and the solvent was removed under vacuum. The resulting residue was washed with diethyl ether (2×10 mL) and vacuum dried. The product was then recrystallized from dichloromethane/n-hexane, affording dark-orange crystals. Yield: 14%.

¹H NMR [(CD₃)₂CO, Me₄Si, δ/ppm]: 9.91 (d, 2H, ³J=5.38 Hz), 9.40 (d, 2H, ³J=8.15 Hz), 9.25 (s, 2H), 8.01 (dd, 2H, ³J=8.19 and 5.41 Hz), 7.25 (m, 3H), 7.16 (td, 6H, ³J=7.44 and 2.04 Hz), 7.06 (m, 6H), 5.98 (m, 2H), 4.85 (m, 2H), 1.62 (s, 3H). ¹³C NMR [(CD₃)₂CO, δ/ppm]: 195.33, 158.56 (d, ¹J=1.83 Hz), 149.08, 147.45, 140.16, 133.72 (d, ²J=10.81 Hz), 133.42, 131.23 (d, ⁴J=2.26 Hz), 131.21 (d, ¹J=44.05 Hz), 130.33, 129.25 (d, ³J=9.89 Hz), 126.85, 87.12, 84.27 (d), 77.80, 27.13. ³¹P NMR [(CD₃)₂CO, δ/ppm]: 50.04 (s). FT-IR [KBr, cm⁻¹]: 3090-3040 (vC—H, aromatic), 2962 (vC—H, aliphatic), 1660 (vC═O), 1481-1430 (vC═C, aromatic), 1405 (vC═N, aromatic), 1386 (C—H, aliphatic), 1282 (vCF₃SO₃). UV-Vis [DCM, λmax/nm (ε/M⁻¹·cm⁻¹)]: 293 (2.47×10⁴), 253 (4.98×10⁴), 370 (6.00×10³). EA (%) found: C 56.3, H 3.5, N 6.6, S 3.9. Calculated for C₄₀H₃₀RuF₃N₄O₄PS (851.79 g/mol): C 56.4, H 3.6, N 6.6, S 3.8.

### Example 8. Synthesis of (2,2′-bipyridyl)(triphenylphosphane)(η⁵-ethyl cyclopentadienylketone)ruthenium(II) trifluoromethanesulfonate ([Ru(CpCOCH₂CH₃)(PPh₃)(bipy)][CF₃SO₃]), complex 8

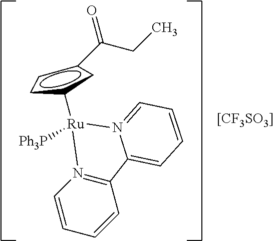

Silver trifluoromethanesulfonate (0.13 g, 0.5 mmol) and 2,2′-bipyridine (0.08 g, 0.5 mmol) were added to a stirring solution of [Ru(CpCOCH₂CH₃)(PPh₃)₂Cl] (complex 2, 0.39 g, 0.5 mmol) in methanol (20 mL). The mixture was refluxed for 4 h in dark, turning from brick-red to brown-red. Then, the solution was separated from the AgCl precipitate by cannula filtration and the solvent was removed under vacuum. The resulting residue was washed with n-hexane (2×10 mL), diethyl ether (2×10 mL), and vacuum dried. The product was then recrystallized from dichloromethane/diethyl ether, affording orange crystals. Yield: 63%.

¹H NMR [(CD₃)₂CO, Me₄Si, δ/ppm]: 9.39 (d, 2H), 8.22 (d, 2H, ³J=7.69 Hz), 7.96 (t, 2H, ³J=7.41 Hz), 7.44 (m, 5H), 7.36 (m, 6H), 7.11 (t, 6H), 5.75 (br, 2H), 4.71 (br, 2H), 2.05 (br, overlapped with residual solvent signal), 0.52 (t, 3H, ³J=6.04 Hz). ¹³C NMR [(CD₃)₂CO, δ/ppm]: 198.61, 157.19, 156.69, 138.00, 133.92 [d, ²J=10.86 Hz], 132.00 [d, ¹J=43.88 Hz], 131.32 [d, ⁴J=1.92 Hz], 129.54 [d, ³J=9.83 Hz], 126.75, 124.35, 86.69, 84.26, 77.87, 32.98, 8.06. ³¹P NMR [(CD₃)₂CO, δ/ppm]: 49.74 (s). FT-IR [KBr, cm^{-1]: 3112}-3052 (vC—H, aromatic), 2967-2873 (vC—H, aliphatic), 1654 (vC═O), 1481-1369 (vC═N+vC═C, aromatic+δC—H, aliphatic), 1259 (vCF₃SO₃), 1224-694 (C—H, aromatic). UV-Vis [DCM, λmax/nm (ε/M⁻¹·cm⁻¹)]: 289 (1.86×10⁴), 345 (sh), 407 (3.72×10³), 455 (sh). EA (%) found: C 56.2, H 4.1, N 3.5, S 4.0. Calculated for C₃₇H₃₂RuF₃N₂O₄PS (789.77 g/mol): C 56.3, H 4.1, N 3.5, S 4.1.

### Example 9. Synthesis of (2,2′-bipyridyl)(triphenylphosphane)(η⁵-phenylmethyl cyclopentadienylketone)ruthenium(II) trifluoromethanesulfonate ([Ru(CpCOCH₂Ph)(PPh₃)(bipy)][CF₃SO₃]), complex 9

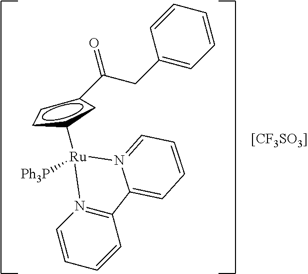

Silver trifluoromethanesulfonate (0.13 g, 0.5 mmol) and 2,2′-bipyridine (0.08 g, 0.5 mmol) were added to a stirring solution of [Ru(CpCOCH₂Ph)(PPh₃)₂Cl](complex 3, 0.42 g, 0.5 mmol) in methanol (20 mL). The mixture was refluxed for 4 h in dark, turning from orange to red. Then, the solution was separated from the AgCl precipitate by cannula filtration and the solvent was removed under vacuum. The resulting residue was washed with n-hexane (2×10 mL), diethyl ether (2×10 mL), and vacuum dried. The product was then recrystallized from dichloromethane/diethyl ether, affording orange crystals. Yield: 52%.

¹H NMR [(CD₃)₂CO, Me₄Si, δ/ppm]: 9.34 (d, 2H, ³J=5.56 Hz), 8.02 (d, 2H, ³J=7.97 Hz), 7.91 (t, 2H, ³J=7.65 Hz), 7.43 (m, 5H), 7.33 (td, 6H, ³J=9.62 Hz), 7.08 (m, 6H), 6.98 (t, 1H, ³J=7.43 Hz), 6.86 (t, 2H, ³J=7.62 Hz), 6.77 (d, 2H, ³J=7.49 Hz), 5.86 (br, 2H), 4.63 (d, 2H), 3.49 (s, 2H). ¹³C NMR [(CD₃)₂CO, δ/ppm]: 194.96, 156.90, 156.53, 137.64, 134.86, 133.94 (d, ²J=10.71 Hz), 131.69, 131.35 (br), 129.51 (d, ³J=9.85 Hz), 129.13, 128.96, 127.03, 126.54, 124.56, 87.90, 84.20, 77.98, 46.30. ³¹P NMR [(CD₃)₂CO, δ/ppm]: 50.21 (s). FT-IR [KBr, cm^{-1]: 3110}-3023 (vC—H, aromatic), 2890 (vC—H, aliphatic), 1672 (vC═O), 1496-1384 (vC═N+vC═C, aromatic+δC—H, aliphatic), 1261 (vCF₃SO₃), 1224-694 (δC—H, aromatic). UV-Vis [DCM, λmax/nm (ε/M⁻¹·cm⁻¹)]: 249 (sh), 290 (1.72×10⁴), 364 (4.56×10³), 410 (sh), 445 (sh). EA (%) found: C 59.2, H 4.0, N 3.3, S 4.0. Calculated for C₄₂H₃₄RuF₃N₂O₄PS (851.84 g/mol): C 59.2, H 4.0, N 3.3, S 3.8.

### Example 10. Synthesis of (4-hydroxy-4′-methoxy-2,2′-bipyridyl)(triphenylphosphane)(η⁵-cyclopentadienyl)ruthenium(II) trifluoromethanesulfonate ([RuCp(PPh₃)(bipy-OH)][CF₃SO₃]), complex 10

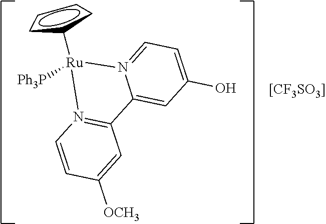

Silver trifluoromethanesulfonate (0.13 g, 0.5 mmol) and 4-hydroxy-4′-methoxy-2,2′-bipyridine (0.10 g, 0.5 mmol) were added to a stirring solution of [RuCp(PPh₃)₂C₁](0.36 g, 0.5 mmol) in methanol (20 mL). The mixture was refluxed for 5 h in dark, turning from orange to dark-orange. Then, the solution was separated from the AgCl precipitate by cannula filtration and the solvent was removed under vacuum. The resulting residue was washed with n-hexane (2×10 mL) and vacuum dried. The product was then recrystallized from dichloromethane/n-hexane, affording yellow-orange crystals. Yield: 94%.

¹H NMR [(CD₃)₂CO, Me₄Si, δ/ppm]: 10.30 (br, 1H), 9.16 (m, 1H), 9.07 (m, 1H), 7.69 (m, 1H), 7.63 (m, 1H), 7.43 (m, 3H), 7.34 (m, 6H), 7.16 (m, 6H), 6.90 (m, 1H), 6.82 (m, 1H), 4.77 (s, 5H), 3.98 (s, 3H). ¹³C NMR [(CD₃)₂CO, δ/ppm]: 167.22, 165.67, 157.68, 157.37, 133.93 (d, ²J=11.18 Hz), 133.40 (d, ¹J=40.64 Hz), 130.77 (d, ⁴J=2.09 Hz), 129.25 (d, ³J=9.44 Hz), 114.58, 113.56, 112.04, 109.43, 78.12 (d, ²J=2.22 Hz), 56.93. ³¹P NMR [(CD₃)₂CO, δ/ppm]: 51.98 (s). FT-IR [KBr, cm⁻¹]: 3500-3300 (vO—H), 3100-3000 (vC—H, aromatic), 2950-2900 (vC—H, aliphatic); 1618-1380 (vC═N+vC═C, aromatic), 1286 (vC-O, aliphatic), 1261 (vCF₃SO₃), 1224-690 (δC—H, aromatic). UV-Vis [DCM, λmax/nm (ε/M⁻¹·cm⁻¹)]: 268 (2.69×10⁴), 290 (sh), 404 (5.46×10³), 455 (sh). EA (%) found: C 54.0, H 3.7, N 3.6, S 4.0. Calculated for C₃₅H₃₀RuF₃N₂O₅PS (779.73 g/mol): C 53.9, H 3.9, N 3.6, S 4.1.

### Example 11. Synthesis of (4-(ethoxycarbonyl)oxy-4′-methoxy-2,2′-bipyridyl)(triphenylphosphane)(η⁵-cyclopentadienyl)ruthenium(II) trifluoromethanesulfonate ([RuCp(PPh₃)(bipy-CE)][CF₃SO₃]), complex 11

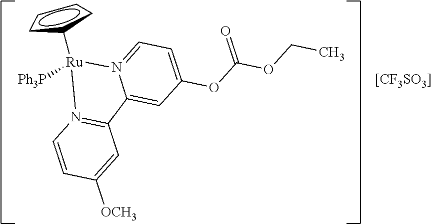

First, N,N-diisopropylethylamine (0.87 mL, 5 mmol) and ethyl chloroformate (0.24 mL, 2.5 mmol) were stepwise added to a stirring solution of 4-hydroxy-4′-methoxy-2,2′-bipyridine (0.10 g, 0.5 mmol) in dichloromethane (20 mL) at 0° C. The mixture was stirred for 30 min in dark at room temperature, turning from colorless to slightly beige. Then, the solvent was removed under vacuum. The product was extracted from the resulting residue by successive solid-liquid extractions firstly with diethyl ether (2×10 mL) and secondly with n-hexane (2×10 mL). The organic phases were collected and the solvents were removed under vacuum, affording the 4-(ethoxycarbonyl)oxy-4′-methoxy-2,2′-bipyridine (bipy-CE) as a white powder. Yield: 97%.

¹H NMR [CDCl₃, Me₄Si, δ/ppm]: 8.62 (d, 1H, ³J=5.44 Hz), 8.42 (d, 1H, ³J=5.64 Hz), 8.24 (m, 1H), 7.92 (m, 1H), 7.17 (m, 1H), 6.82 (m, 1H), 4.31 (q, 2H, ³J=7.13 Hz), 3.90 (s, 3H), 1.35 (t, 3H, ³J=7.12 Hz). ¹³C NMR [CDCl₃, δ/ppm]: 166.68, 158.76, 158.22, 156.73, 152.13, 150.61, 150.18, 116.18, 113.66, 111.03, 106.37, 65.33, 55.34, 14.07. FT-IR [KBr, cm⁻¹]: 3106-2838 (vC—H, aromatic and aliphatic), 1770 (vC═O), 1585-1367 (vC═N+vC═C, aromatic), 1249-1224 (vC—O, aliphatic), 1101-773 (δC—H, aromatic and aliphatic). UV-Vis [DCM, λmax/nm (ε/M⁻¹·cm⁻¹)]: 250 (1.18×10⁴), 277 (1.36×10⁴). EA (%) found: C 59.9, H 5.0, N 9.6. Calculated for C₁₄H₁₄N₂O₄·0.2CH₂Cl₂ (282.76 g/mol): C 58.6, H 5.0, N 9.6.

Afterwards, silver trifluoromethanesulfonate (0.13 g, 0.5 mmol) and 4-(ethoxycarbonyl)oxy-4′-methoxy-2,2′-bipyridine (0.14 g, 0.5 mmol) were added to a stirring solution of [RuCp(PPh₃)₂Cl] (0.36 g, 0.5 mmol) in methanol (20 mL). The mixture was refluxed for 5 h in dark, turning from orange to red. Then, the solution was separated from the AgCl precipitate by cannula filtration and the solvent was removed under vacuum. The resulting residue was washed with n-hexane (2×10 mL) and vacuum dried. The product was then recrystallized from dichloromethane/diethyl ether, affording orange crystals. Yield: 85%.

¹H NMR [(CD₃)₂CO, Me₄Si, δ/ppm]: 9.53 (d, 1H, ³J=6.37 Hz), 9.26 (d, 1H, ³J=6.51 Hz), 8.11 (d, 1H, ³J=2.44 Hz), 7.70 (d, 1H, ³J=2.67 Hz), 7.42 (m, 3H), 7.36 (m, 7H), 7.15 (m, 6H), 6.99 (dd, 1H, ³J=6.53 Hz and ⁴J=2.71 Hz), 4.86 (s, 5H), 4.36 (q, 2H, ³J=7.12 Hz), 3.98 (s, 3H), 1.35 (t, 3H, ³J=7.12 Hz). ¹³C NMR [(CD₃)₂CO, δ/ppm]: 167.24, 158.77, 158.76, 158.49 (d), 157.62 (d), 157.32, 152.45, 133.89 (d, ²J=11.12 Hz), 132.71 (d, ¹J=41.39 Hz), 130.90 (d, ⁴J=2.05 Hz), 129.37 (d, ³J=9.55 Hz), 119.13, 117.20, 113.96, 110.06, 78.73 (d, ²J=2.09 Hz), 66.50, 57.05, 14.31. ³¹P NMR [(CD₃)₂CO, δ/ppm]: 51.52 (s). FT-IR [KBr, cm⁻¹]: 3100-3050 (vC—H, aromatic), 3000-2840 (vC—H, aliphatic), 1770 (vC═O), 1616-1384 (vC═N+vC═C, aromatic), 1290-1276 (vC—O, aliphatic), 1260 (vCF₃SO₃), 1209-698 (C—H, Cp, aromatic and aliphatic). UV-Vis [DCM, λmax/nm (ε/M⁻¹·cm⁻¹)]: 279 (2.37×10⁴), 325 (sh), 355 (sh), 425 (4.53×10³), 475 (sh). EA (%) found: C 53.1, H 3.7, N 3.3, S 3.5. Calculated for C₃₈H₃₄RuF₃N₂O₇PS (851.80 g/mol): C 53.5, H 4.0, N 3.3, S 3.7.

### Example 12. Alternative synthesis of (4-(ethoxycarbonyl)oxy-4′-methoxy-2,2′-bipyridyl)(triphenylphosphane)(η⁵-cyclopentadienyl)ruthenium(II) trifluoromethanesulfonate ([RuCp(PPh₃)(bipy-CE)][CF₃SO₃]), complex 11

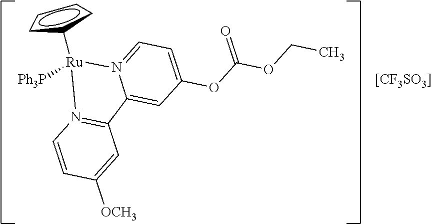

N,N-diisopropylethylamine (0.44 mL, 2.5 mmol) and ethyl chloroformate (0.12 mL, 1.25 mmol) were stepwise added to a stirring solution of [RuCp(PPh₃)(bipy-OH)][CF₃SO₃] (complex 10, 0.19 g, 0.25 mmol) in dichloromethane (10 mL) at 0° C. The mixture was stirred for 4 h in dark at room temperature, turning from yellow-orange to red. Then, the solvent was removed under vacuum. The resulting residue was washed with n-hexane (2×10 mL) and vacuum dried. The product was then recrystallized from dichloromethane/n-hexane, affording orange crystals. Yield: 88%.

¹H NMR [(CD₃)₂CO, Me₄Si, δ/ppm]: 9.53 (d, 1H, ³J=6.37 Hz), 9.26 (d, 1H, ³J=6.51 Hz), 8.11 (d, 1H, ³J=2.44 Hz), 7.70 (d, 1H, ³J=2.67 Hz), 7.42 (m, 3H), 7.36 (m, 7H), 7.15 (m, 6H), 6.99 (dd, 1H, ³J=6.53 Hz and ⁴J=2.71 Hz), 4.86 (s, 5H), 4.36 (q, 2H, ³J=7.12 Hz), 3.98 (s, 3H), 1.35 (t, 3H, ³J=7.12 Hz). ¹³C NMR [(CD₃)₂CO, δ/ppm]: 167.24, 158.77, 158.76, 158.49 (d), 157.62 (d), 157.32, 152.45, 133.89 (d, ²J=11.12 Hz), 132.71 (d, ¹J=41.39 Hz), 130.90 (d, ⁴J=2.05 Hz), 129.37 (d, ³J=9.55 Hz), 119.13, 117.20, 113.96, 110.06, 78.73 (d, ²J=2.09 Hz), 66.50, 57.05, 14.31. ³¹P NMR [(CD₃)₂CO, δ/ppm]: 51.52 (s).

### Example 13. Synthesis of (4-methoxy-4′-(4-oxopentanoate)-2,2′-bipyridyl)(triphenylphosphane)(η⁵-cyclopentadienyl)ruthenium(II) trifluoromethanesulfonate ([RuCp(PPh₃)(bipy-LEV)][CF₃SO₃]), complex 12

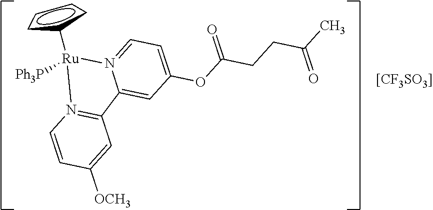

Levulinic acid (0.03 mL, 0.3 mmol), 1-ethyl-3-(3-dimethylaminopropyl)carbodiimide (0.06 g, 0.3 mmol) and 4-dimethylaminopyridine (0.02 g; 0.2 mmol) were added to a stirring solution of [RuCp(PPh₃)(bipy-OH)][CF₃SO₃] (complex 10, 0.16 g, 0.2 mmol) in tetrahydrofuran (10 mL). The mixture was stirred for 1 h in dark at room temperature, turning from yellow-orange to red. Then, the solvent was removed under vacuum. The resulting residue was washed with n-hexane (2×10 mL), diethyl ether (2×10 mL), and vacuum dried. The product was then recrystallized from dichloromethane/n-hexane, affording orange crystals.

¹H NMR [CDCl₃, Me₄Si, δ/ppm]: 8.94 (d, 1H, ³J=6.85 Hz), 8.74 (d, 1H, ³J=6.57 Hz), 8.18 (m, 1H), 7.60 (m, 1H), 7.38 (m, 3H), 7.28 (m, 6H), 6.99 (m, 7H), 6.74 (m, 1H), 4.61 (s, 5H), 4.00 (s, 3H), 2.96 (m, 2H), 2.92 (m, 2H), 2.24 (s, 3H). ³¹P NMR [CDCl₃, δ/ppm]: 51.46 (s).

### D) Synthesis of Peptides

### Example 14. Synthesis of NH₂-¹valine-²serine-³proline-⁴proline-⁵leucine-⁶threonine-⁷leucine-⁸glycine-⁹glutamine-¹⁰leucine-¹¹leucine-¹²serine-CONH₂ (VSPPLTLGQLLS), peptide P1

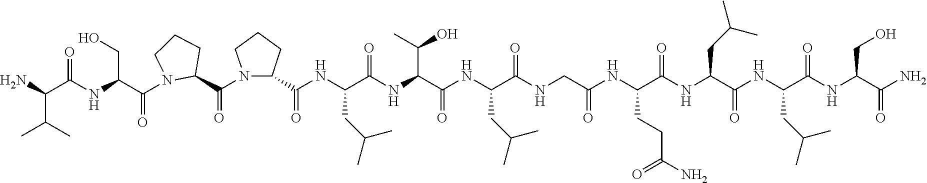 [SMILES: CC(C)C[C@H](NC(=O)[C@H](CC(C)C)NC(=O)[C@H](CCC(N)=O)NC(=O)CNC(=O)[C@H](CC(C)C)NC(=O)[C@@H](NC(=O)[C@H](CC(C)C)NC(=O)[C@H]1CCCN1C(=O)[C@@H]1CCCN1C(=O)[C@H](CO)NC(=O)[C@H](N)C(C)C)[C@@H](C)O)C(=O)N[C@@H](CO)C(N)=O]

The peptide VSPPLTLGQLLS (P1) was synthesized as C-terminal amide by ultrasound-assisted solid phase peptide synthesis on a rink-amide 4-methylbenzhydrylamine resin (100-200 mesh, 0.78 mmol/g loading) at 0.3 mmol scale, using fmoc-L-amino acids protected in their side chains with standard trifluoroacetic acid-labile groups, following stepwise cycles of amino acid coupling to the resin and N-terminal fmoc removal from the coupled amino acid. The coupling steps were performed under ultrasonication (37 kHz, 30° C.) using 5-fold excess of each amino acid (1.5 mmol) in the presence of an equivalent amount of hexafluorophosphate benzotriazole tetramethyl uronium (569 mg, 1.5 mmol) and 10-fold excess of N,N-diisopropylethylamine (523 μL, 3 mmol) in dimethylformamide (3 mL), during variable periods of time according to Table 1. The removal of the fmoc group after each coupling step was achieved by treating the resin with a solution of piperidine in dimethylformamide (20% v/v, 3 mL) for 5 min under ultrasonication (37 kHz, 30° C.). A Kaiser test was performed at each step of amino acid coupling and fmoc removal to estimate the completeness of the reactions (Kaiser, E. et. al, 1970). After coupling all the amino acids and removing the fmoc groups, the resin was treated with a mixture of trifluoroacetic acid/triisopropylsilane/water (95:2.5:2.5, 5 mL) for 2 h at room temperature, to promote the cleavage and full deprotection of the peptide. Then, the solution was collected and concentrated until 1 mL under a stream of dinitrogen gas. The addition of 15-fold excess of ice-cold diethyl ether produced a precipitate that was decanted upon centrifugation (5000 rpm, 5 min, 4° C.) and washed with diethyl ether (2×10 mL). The product was purified by semi-preparative RP-HPLC (method 1) and the fractions with satisfactory purity (>97%) were collected and Lyophilized overnight, affording a white powder.

RP-HPLC (method 4): t_{R}=15.9 mi. ESI-MS (positive mode, m/z) found: 1223.8 [M+H]⁺, 612.5 [M+2H]²⁺. Calculated for C₅₆H₉₈N₁₄O₁₆ (1222.72 u): 1223.7 [M+H]⁺, 612.4 [M+2H]²⁺.

TABLE 1
Conditions for the coupling of each amino acid
to the resin during the synthesis of P1.

| Order of | | Quantity | |
|---|---|---|---|
| coupling to | Amino | equivalent to 1.5 | Coupling |
| the resin | acid | mmol (mg) | time (min) |
| 1 | ¹²serine | 575 | 15 |
| 2 | ¹¹leucine | 530 | 10 |
| 3 | ¹⁰leucine | 530 | 10 |
| 4 | ⁹glutamine | 916 | 20 |
| 5 | ⁸glycine | 446 | 5 |
| 6 | ⁷leucine | 530 | 15 |
| 7 | ⁶threonine | 596 | 20 |
| 8 | ⁵leucine | 530 | 15 |
| 9 | ⁴proline | 506 | 20 |
| 10 | ³proline | 506 | 25 |
| 11 | ²serine | 575 | 15 |
| 12 | ¹valine | 509 | 15 |

### Example 15. Synthesis of 4-oxopetanoyl-NH-¹valine-²serine-³proline-⁴proline-⁵leucine-⁶threonine-⁷leucine-⁸glycine-⁹glutamine-¹⁰leucine-¹¹leucine-¹²serine-CONH₂ (CH₃COC₂H₄CONH-VSPPLTLGQLLS), Peptide P2

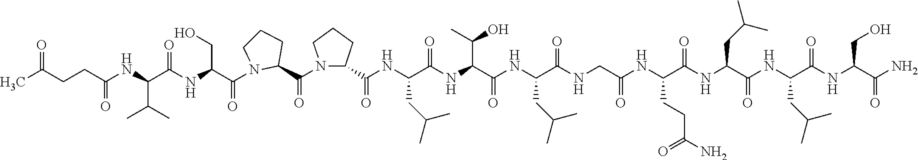 [SMILES: CC(=O)CCC(=O)N[C@@H](C(=O)N[C@@H](CO)C(=O)N1CCC[C@H]1C(=O)N1CCC[C@@H]1C(=O)N[C@@H](CC(C)C)C(=O)N[C@H](C(=O)N[C@@H](CC(C)C)C(=O)NCC(=O)N[C@@H](CCC(N)=O)C(=O)N[C@@H](CC(C)C)C(=O)N[C@@H](CC(C)C)C(=O)N[C@@H](CO)C(N)=O)[C@@H](C)O)C(C)C]

The peptide CH₃COC₂H₄CONH-VSPPLTLGQLLS (P2) was synthesized as C-terminal amide by ultrasound-assisted solid phase peptide synthesis on a rink-amide 4-methylbenzhydrylamine resin (100-200 mesh, 0.78 mmol/g loading) at 0.3 mmol scale, using fmoc-L-amino acids protected in their side chains with standard trifluoroacetic acid-labile groups, following stepwise cycles of amino acid coupling to the resin and N-terminal fmoc removal from the coupled amino acid. The twelve amino acids (12serine→1valine) were coupled to the resin and the fmoc groups were removed as described for the peptide VSPPLTLGQLLS (P1) in example 14. After coupling the last amino acid (1valine) and removing the fmoc group, the resin was treated with 5-fold excess of levulinic acid (154 μL, 1.5 mmol) in the presence of an equivalent amount of hexafluorophosphate benzotriazole tetramethyl uronium (569 mg, 1.5 mmol) and 10-fold excess of N,N-diisopropylethylamine (523 μL, 3 mmol) in dimethylformamide (3 mL), for 15 min under ultrasonication (37 kHz, 30° C.). The completeness of this reaction was estimated by the Kaiser test as performed for the amino acid coupling and fmoc removal steps (Kaiser, E. et. al, 1970). Then, the resin was treated with a mixture of trifluoroacetic acid/triisopropylsilane/water (95:2.5:2.5, 5 mL) for 2 h at room temperature, to promote the cleavage and full deprotection of the peptide. The solution was collected and concentrated until 1 mL under a stream of dinitrogen gas. The addition of 15-fold excess of ice-cold diethyl ether produced a precipitate that was decanted upon centrifugation (5000 rpm, 5 min, 4° C.) and washed with diethyl ether (2×10 mL). The product was purified by semi-preparative RP-HPLC (method 1) and the fractions with satisfactory purity (>97%) were collected and lyophilized overnight, affording a white powder.

RP-HPLC: t_{R}=16.0 min (method 4), t_{R}=12.2 min (method 5). ESI-MS (positive mode, m/z) found: 1322.2 [M+H]⁺, 661.4 [M+2H]²⁺. Calculated for C₆₁H₁₀₄N₁₄O₁₈ (1320.77 u): 1321.8 [M+H]⁺, 661.4 [M+2H]²⁺.

### Example 16. Synthesis of 4-hydrazineyl-4-oxobutanoyl-NH-¹valine-²serine-³proline-⁴proline-⁵leucine-⁶threonine-⁷leucine-⁸glycine-⁹glutamine-¹⁰leucine-¹¹leucine-¹²serine-CONH₂ (N₂H₃COC₂H₄CONH-VSPPLTLGQLLS), peptide P3

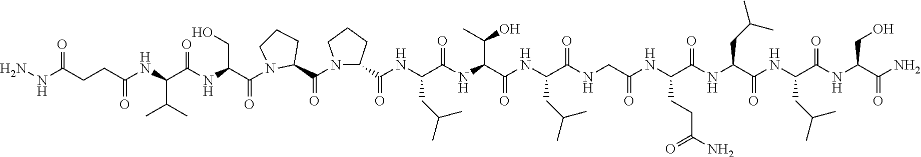 [SMILES: CC(C)C[C@H](NC(=O)[C@H](CC(C)C)NC(=O)[C@H](CCC(N)=O)NC(=O)CNC(=O)[C@H](CC(C)C)NC(=O)[C@@H](NC(=O)[C@H](CC(C)C)NC(=O)[C@H]1CCCN1C(=O)[C@@H]1CCCN1C(=O)[C@H](CO)NC(=O)[C@H](NC(=O)CCC(=O)NN)C(C)C)[C@@H](C)O)C(=O)N[C@@H](CO)C(N)=O]

The peptide N₂H₃COC₂H₄CONH-VSPPLTLGQLLS (P3) was synthesized as C-terminal amide by ultrasound-assisted solid phase peptide synthesis on a rink-amide 4-methylbenzhydrylamine resin (100-200 mesh, 0.78 mmol/g loading) at 0.3 mmol scale, using fmoc-L-amino acids protected in their side chains with standard trifluoroacetic acid-labile groups, following stepwise cycles of amino acid coupling to the resin and N-terminal fmoc removal from the coupled amino acid. The twelve amino acids (12serine→1valine) were coupled to the resin and the fmoc groups were removed as described for the peptide VSPPLTLGQLLS (P1) in example 14. After coupling the last amino acid (1valine) and removing the fmoc group, the resin was treated with an equivalent amount of succinic anhydride (30 mg, 0.3 mmol) and 2-fold excess of N,N-diisopropylethylamine (105 μL, 0.6 mmol) in dimethylformamide (3 mL), for 7 min under ultrasonication (37 kHz, 30° C.). Then, the resin was treated with 5-fold excess of fmoc-hydrazine (381 mg, 1.5 mmol) in the presence of an equivalent amount of hexafluorophosphate benzotriazole tetramethyl uronium (569 mg, 1.5 mmol) and 10-fold excess of N,N-diisopropylethylamine (523 μL, 3 mmol) in dimethylformamide (3 mL), for 20 min under ultrasonication (37 kHz, 30° C.). The fmoc group was removed by reaction with a solution of piperidine in dimethylformamide (20% v/v, 3 mL) for 5 min under ultrasonication (37 kHz, 30° C.). The completeness of these reactions was estimated by the Kaiser test as performed for the amino acid coupling and fmoc removal steps (Kaiser, E. et. al, 1970). Then, the resin was treated with a mixture of trifluoroacetic acid/triisopropylsilane/water (95:2.5:2.5, 5 mL) for 2 h at room temperature, to promote the cleavage and full deprotection of the peptide. The solution was collected and concentrated until 1 mL under a stream of dinitrogen gas. The addition of 15-fold excess of ice-cold diethyl ether produced a precipitate that was decanted upon centrifugation (5000 rpm, 5 min, 4° C.) and washed with diethyl ether (2×10 mL). The product was purified by semi-preparative RP-HPLC (method 2) and the fractions with satisfactory purity (≥97%) were collected and lyophilized overnight, affording a white powder.

RP-HPLC: t_{R}=15.9 min (method 4), t_{R}=12.8 min (method 5). ESI-MS (positive mode, m/z) found: 1338.2 [M+H]⁺, 669.7 [M+2H]²⁺. Calculated for C₆₀H₁₀₄N₁₆O₁₈ (1336.77 u): 1337.8 [M+H]⁺, 669.4 [M+2H]²⁺.

### Example 17. Synthesis of 4-hydrazineyl-4-oxobutanoic 4-oxobutanoyl anhydride-NH-¹valine-²serine-³proline-⁴proline-⁵leucine-⁶threonine-⁷leucine-⁸ glycine-⁹glutamine-¹⁰1leucine-¹¹leucine-¹²serine-CONH₂ (N₂H₃CO(C₂H₄CO)₂OCONH-VSPPLTLGQLLS), peptide P4

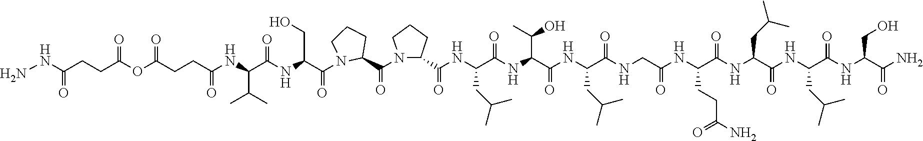 [SMILES: CC(C)C[C@H](NC(=O)[C@H](CC(C)C)NC(=O)[C@H](CCC(N)=O)NC(=O)CNC(=O)[C@H](CC(C)C)NC(=O)[C@@H](NC(=O)[C@H](CC(C)C)NC(=O)[C@H]1CCCN1C(=O)[C@@H]1CCCN1C(=O)[C@H](CO)NC(=O)[C@H](NC(=O)CCC(=O)OC(=O)CCC(=O)NN)C(C)C)[C@@H](C)O)C(=O)N[C@@H](CO)C(N)=O]

The peptide N₂H₃CO(C₂H₄CO)₂OCONH-VSPPLTLGQLLS (P4) was synthesized as C-terminal amide by ultrasound-assisted solid phase peptide synthesis on a rink-amide 4-methylbenzhydrylamine resin (100-200 mesh, 0.78 mmol/g loading) at 0.3 mmol scale, using fmoc-L-amino acids protected in their side chains with standard trifluoroacetic acid-labile groups, following stepwise cycles of amino acid coupling to the resin and N-terminal fmoc removal from the coupled amino acid. The twelve amino acids (12serine→1valine) were coupled to the resin and the fmoc groups were removed as described for the peptide VSPPLTLGQLLS (P1) in example 14. After coupling the last amino acid (1valine) and removing the fmoc group, the resin was treated with 5-fold excess of succinic anhydride (150 mg, 1.5 mmol) and 10-fold excess of N,N-diisopropylethylamine (523 μL, 3 mmol) in dimethylformamide (3 mL), for 20 min under ultrasonication (37 kHz, 30° C.). Then, the resin was treated with 5-fold excess of fmoc-hydrazine (381 mg, 1.5 mmol) in the presence of an equivalent amount of hexafluorophosphate benzotriazole tetramethyl uronium (569 mg, 1.5 mmol) and 10-fold excess of N,N-diisopropylethylamine (523 μL, 3 mmol) in dimethylformamide (3 mL), for 20 min under ultrasonication (37 kHz, 30° C.). The fmoc group was removed by reaction with a solution of piperidine in dimethylformamide (20% v/v, 3 mL) for 5 min under ultrasonication (37 kHz, 30° C.). The completeness of these reactions was estimated by the Kaiser test as performed for the amino acid coupling and fmoc removal steps (Kaiser, E. et. al, 1970). Then, the resin was treated with a mixture of trifluoroacetic acid/triisopropylsilane/water (95:2.5:2.5, 5 mL) for 2 h at room temperature, to promote the cleavage and full deprotection of the peptide. The solution was collected and concentrated until 1 mL under a stream of dinitrogen gas. The addition of 15-fold excess of ice-cold diethyl ether produced a precipitate that was decanted upon centrifugation (5000 rpm, 5 min, 4° C.) and washed with diethyl ether (2×10 mL). The product was purified by semi-preparative RP-HPLC (method 2) and the fractions with satisfactory purity (97%) were collected and lyophilized overnight, affording a white powder.

RP-HPLC (method 4): t_{R}=16.1 min. ESI-MS (positive mode, m/z) found: 1436.1 [M+H]⁺, 718.8 [M+2H]²⁺. Calculated for C₆₄H₁₀₈N₁₆O₂₁ (1436.79 u): 1437.8 [M+H]⁺, 719.4 [M+2H]²⁺.

### E) Synthesis of Controlled-Release Multi-Functional Drug (Ruthenium-Peptide Conjugates)

### Example 18. Synthesis of (2,2′-bipyridyl)(triphenylphosphane)(4-((E,Z)-2-η⁵-cyclopentadienylformylhydrazineylidene)pentanoyl-VSPPLTLGQLLS)ruthenium(II) trifluoromethanesulfonate ([Ru(CpCON₂HCCH₃C₂H₄CONH-VSPPLTLGQLLS)(PPh)(bipy)][CF₃SO₃]), conjugate RuPC1

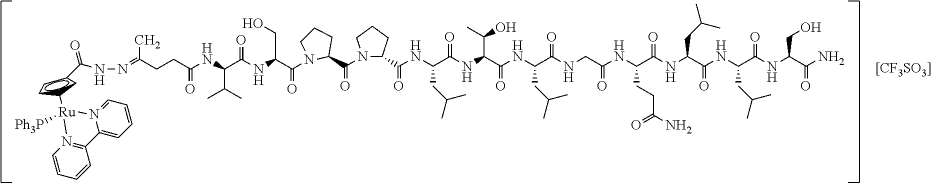

Trifluoroacetic acid (20 μL, 0.2% v/v) and [Ru(CpCONHNH₂)(PPh₃)(bipy)][CF₃SO₃] (complex 4, 39 mg, 50 μmol) were added to a stirring solution of CH₃COC₂H₄CONH-VSPPLTLGQLLS (peptide P2, 66 mg, 50 μmol) in methanol (10 mL). The mixture turned from colorless to orange and was stirred overnight at room temperature. Then, the solvent was removed under vacuum. The resulting residue was washed with ether (2×10 mL) and vacuum dried. The product was purified by semi-preparative RP-HPLC (method 3) and the fractions with satisfactory purity (97%) were collected and lyophilized overnight, affording an orange powder.

RP-HPLC (method 5): t_{R}=19.6 min. ESI-MS (positive mode, m/z) found: 973.7 [M+H]²⁺. Calculated for C₉₅H₁₃₂RuN₁₈O₁₈P (1945.87 u): 973.4 [M+H]²⁺.

### Example 19. Synthesis of (2,2′-bipyridyl)(triphenylphosphane)(4-((E,Z)-2-(1-η⁵-cyclopentadienylethylidene)hydrazineyl)-4-oxobutanoyl-VSPPLTLGQLLS)ruthenium(II) trifluoromethanesulfonate ([Ru(CpCCH₃N₂HCOC₂H₄CONH-VSPPLTLGQLLS)(PPh)(bipy)][CF₃SO₃]), conjugate RuPC2

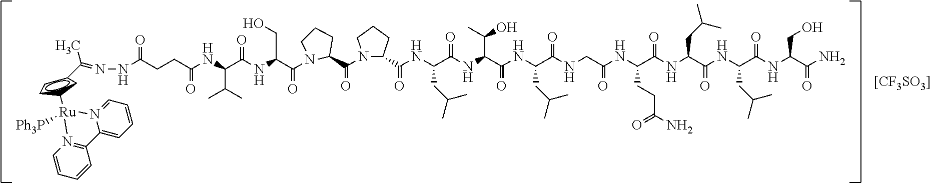

Trifluoroacetic acid (20 μL, 0.2% v/v) and [Ru(CpCOCH₃)(PPh₃)(bipy)][CF₃SO₃] (complex 5, 39 mg, 50 μmol) were added to a stirring solution of N₂H₃COC₂H₄CONH-VSPPLTLGQLLS (peptide P3, 67 mg, 50 μmol) in methanol (10 mL). The mixture turned from colorless to orange and was stirred overnight at room temperature. Then, the solvent was removed under vacuum. The resulting residue was washed with ether (2×10 mL) and vacuum dried. The product was purified by semi-preparative RP-HPLC (method 3) and the fractions with satisfactory purity (>97%) were collected and lyophilized overnight, affording an orange powder.

RP-HPLC (method 5): t_{R}=21.1 min. ESI-MS (positive mode, m/z) found: 973.3 [M+H]²⁺. Calculated for C₉₅H₁₃₂RuN₁₈O₁₈P (1945.87 u): 973.4 [M+H]²⁺.

### Example 20. Synthesis of (1,10-phenanthroline)(triphenylphosphane)(4-((E,Z)-2-(1-η⁵-cyclopentadienylethylidene)hydrazineyl)-4-oxobutanoyl-VSPPLTLGQLLS)ruthenium(II) trifluoromethanesulfonate ([Ru(CpCCH₃N₂HCOC₂H₄CONH-VSPPLTLGQLLS)(PPh₃)(phen)][CF₃SO₃]), conjugate RuPC3

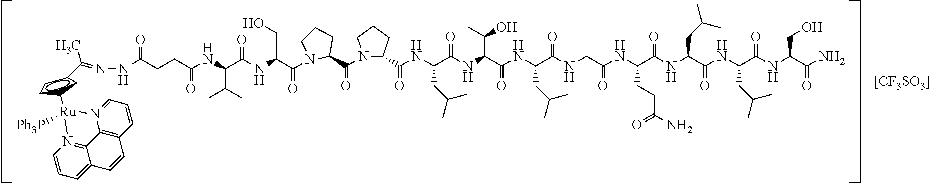

Trifluoroacetic acid (20 μL, 0.2% v/v) and [Ru(CpCOCH₃)(PPh₃)(phen)][CF₃SO₃] (complex 6, 40 mg, 50 μmol) were added to a stirring solution of N₂H₃COC₂H₄CONH-VSPPLTLGQLLS (peptide P3, 67 mg, 50 μmol) in methanol (10 mL). The mixture turned from colorless to orange and was stirred overnight at room temperature. Then, the solvent was removed under vacuum. The resulting residue was washed with ether (2×10 mL) and vacuum dried. The product was purified by semi-preparative RP-HPLC (method 3) and the fractions with satisfactory purity (>97%) were collected and lyophilized overnight, affording an orange powder.

RP-HPLC (method 5): t_{R}=22.8 min. ESI-MS (positive mode, m/z) found: 1970.2 [M]⁺, 985.9 [M+H]2⁺. Calculated for C₉₇H₁₃₂RuN₁₈O₁₈P (1969.87 u): 1969.9 [M]⁺, 985.5 [M+H]²⁺.

### Stability Studies

The stability of the complexes 4-11 in organic and aqueous media was evaluated over 24 h by UV-Vis spectroscopy at room temperature, in dark, on a Jasco V-660 spectrometer using quartz cuvettes (1 cm optical path length). The electronic spectra (270-900 nm) were acquired at regular periods of time (0 h, 0.25 h, 0.5 h, 0.75 h, 1 h, 2 h, 3 h, 4 h, 5 h, 6 h, 24 h) for solutions of 100% dimethyl sulfoxide (complexes 4-11) and 5% dimethyl sulfoxide/95% Dulbecco's Modified Eagle Medium (complexes 4, 5 and 8-11) or 10% dimethyl sulfoxide/90% Dulbecco's Modified Eagle Medium (complexes 6 and 7) at concentrations ranging from 7.0×10-5 M to 3.0×10-4 M.

### G) Results of the Stability Studies

### Example 21. Evaluation of the Stability of the Complexes 4-11 in Organic Media

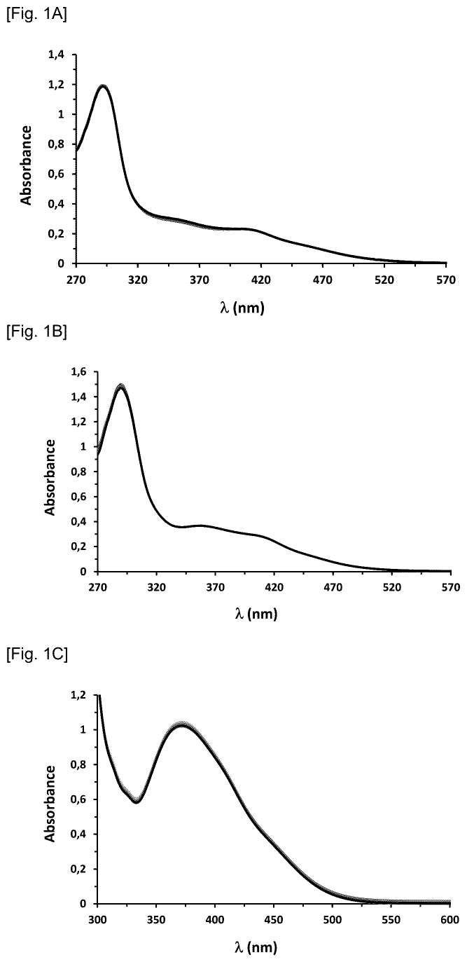

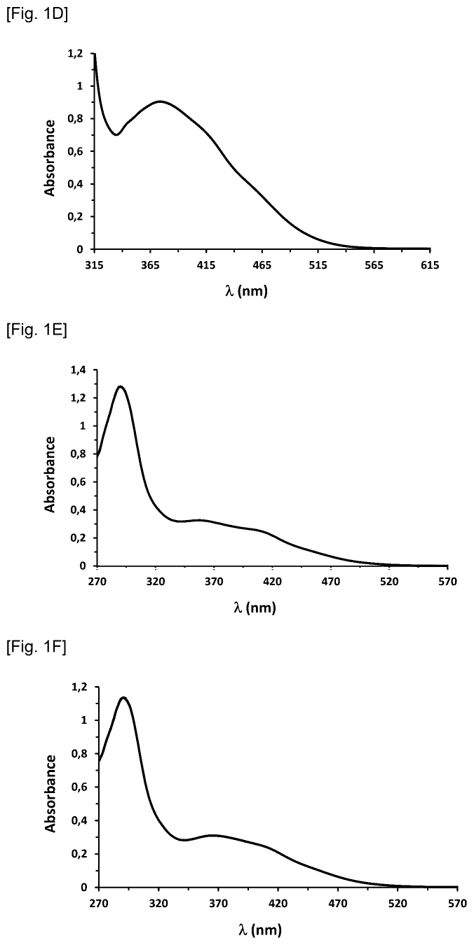

The complexes 4-11 showed to be stable in dimethyl sulfoxide (co-solvent used in the biological studies) for at least 24 h, as no significant changes were observed in the UV-Vis spectra over this period (FIGS. 1A to H) and the respective relative variation of the maximum absorbance of the π→π* transitions (289-300 nm) and charge transfer bands (357-425 nm) was <4% (Tables 2a and 2b).

TABLE 2a
Relative variation (%) of the maximum absorbance of the π →
π * transitions and charge transfer bands in the UV-Vis
spectra of the complexes 4-7 in dimethyl sulfoxide over 24 h.

| Time | Complex (λₘₐₓ/nm) | | | | | |
|---|---|---|---|---|---|---|
| (h) | 4 (291) | 4 (406) | 5 (289) | 5 (357) | 6 (372) | 7 (374) |
| 0 | 0.0 | 0.0 | 0.0 | 0.0 | 0.0 | 0.0 |
| 0.25 | 0.3 | 0.0 | 0.3 | 0.1 | −0.1 | 0.0 |
| 0.5 | 0.3 | −0.1 | −0.5 | 0.0 | 0.2 | −0.1 |
| 0.75 | 0.3 | −0.2 | −0.4 | 0.2 | 0.0 | −0.1 |
| 1 | 0.5 | 0.1 | −0.3 | 0.1 | 0.9 | −0.1 |
| 2 | 0.4 | −0.2 | −0.9 | 0.3 | 2.6 | −0.3 |
| 3 | 0.6 | 0.2 | −1.2 | 0.3 | 1.3 | −0.2 |
| 4 | 0.7 | 0.9 | −1.2 | 0.3 | 1.5 | −0.5 |
| 5 | −0.3 | 1.0 | −1.5 | 0.5 | −0.2 | −0.3 |
| 6 | −0.2 | 0.6 | −1.5 | 0.2 | −0.3 | −0.4 |
| 24 | −0.2 | 1.6 | −1.9 | 0.3 | 0.3 | −0.3 |

TABLE 2b
Relative variation (%) of the maximum absorbance of the π →
π * transitions and charge transfer bands in the UV-Vis
spectra of the complexes 8-11 in dimethyl sulfoxide over 24 h.

| | Complex (λₘₐₓ/nm) | | | | | | |
|---|---|---|---|---|---|---|---|
| Time | 8 | 8 | 9 | 9 | 10 | 10 | 11 |
| (h) | (290) | (358) | (291) | (365) | (300) | (403) | (425) |
| 0 | 0.0 | 0.0 | 0.0 | 0.0 | 0.0 | 0.0 | 0.0 |
| 0.25 | 0.0 | 0.0 | 0.0 | 0.2 | 0.0 | 0.0 | 0.0 |
| 0.5 | 0.2 | 0.1 | −0.2 | 0.2 | 0.0 | −0.2 | 0.2 |
| 0.75 | 0.0 | 0.0 | −0.3 | 0.2 | −0.1 | −0.3 | 0.1 |
| 1 | 0.1 | 0.3 | −0.3 | 0.3 | 0.9 | 0.7 | 0.3 |
| 2 | 0.0 | 0.2 | −0.3 | 0.6 | 1.5 | 1.5 | 0.1 |
| 3 | −0.3 | 0.0 | −0.1 | 0.9 | 2.5 | 2.5 | 0.0 |
| 4 | −0.1 | 0.0 | −0.1 | 0.6 | 3.0 | 3.1 | −0.2 |
| 5 | −0.1 | −0.3 | −0.3 | 0.4 | 3.0 | 3.2 | −0.3 |
| 6 | −0.2 | 0.3 | −0.2 | 0.7 | 2.8 | 3.1 | −0.3 |
| 24 | −0.2 | 0.2 | −0.1 | 0.8 | 3.3 | 4.2 | 0.2 |

### Example 22. Evaluation of the Stability of the Complexes 4-11 in Aqueous Media

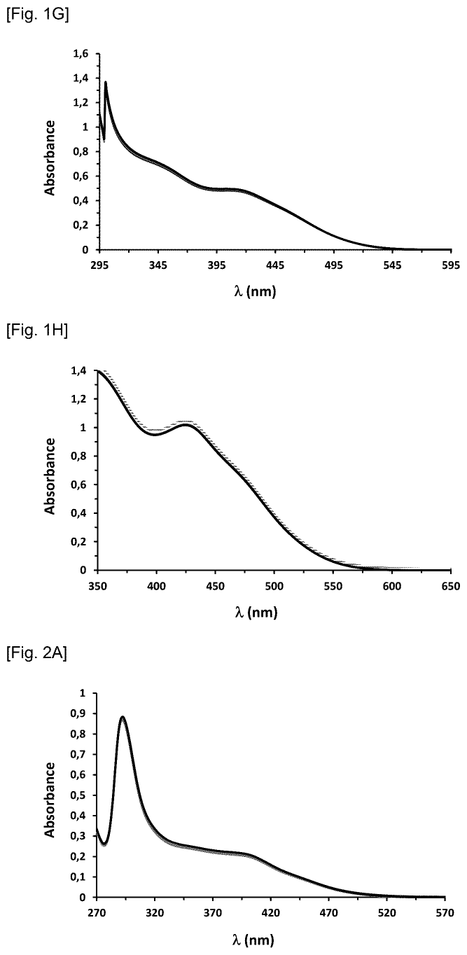

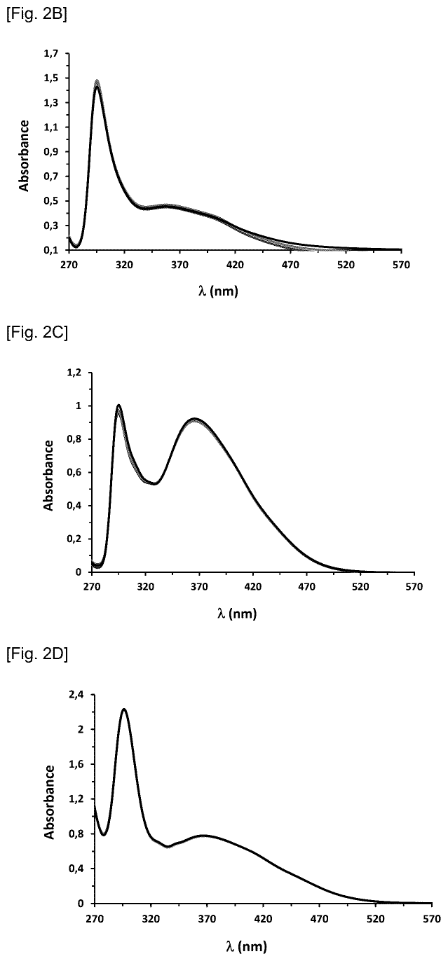

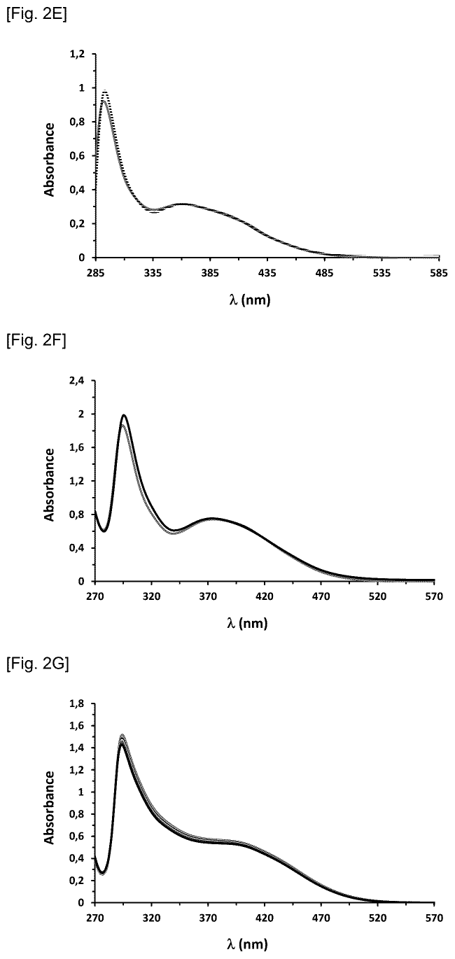

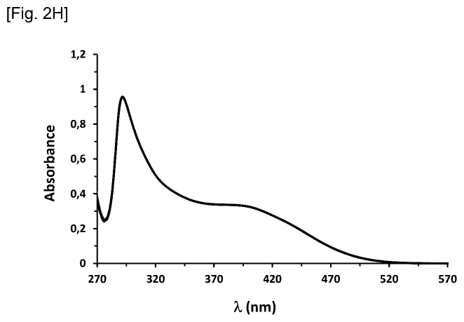

The complexes 4-11 showed to be stable in a solution of 95% or 90% Dulbecco's Modified Eagle Medium (cell culture medium used in the biological studies) and 5% or 10% dimethyl sulfoxide for at least 24 h, as no significant changes were observed in the UV-Vis spectra over this period (FIGS. 2A to H) and the respective relative variation of the maximum absorbance of the π→π* transitions (292-296 nm) and charge transfer bands (358-400 nm) was <7% (Tables 3a and 3b).

TABLE 3a
Relative variation (%) of the maximum absorbance of the
π → π * transitions and charge transfer
bands in the UV-Vis spectra of the complexes 4 and 5 in
5% dimethyl sulfoxide/95% Dulbecco's Modified Eagle
Medium and of the complexes 6 and 7 in 10% dimethyl sulfoxide/90%
Dulbecco's Modified Eagle Medium over 24 h.

| | Complex (λₘₐₓ/nm) | | | | | | | |
|---|---|---|---|---|---|---|---|---|
| Time | 4 | 4 | 5 | 5 | 6 | 6 | 7 | 7 |
| (h) | (292) | (395) | (295) | (358) | (295) | (366) | (296) | (367) |
| 0 | 0.0 | 0.0 | 0.0 | 0.0 | 0.0 | 0.0 | 0.0 | 0.0 |
| 0.25 | 0.1 | −0.2 | 0.6 | −1.0 | −0.4 | 0.0 | 0.0 | 0.3 |
| 0.5 | 0.1 | −0.7 | −0.1 | −2.1 | 0.4 | 0.4 | 0.1 | 0.4 |
| 0.75 | −0.4 | −1.3 | 0.3 | −2.7 | 0.3 | 0.3 | 0.3 | 0.6 |
| 1 | −0.3 | −0.9 | 0.0 | −3.4 | −0.2 | 0.0 | 0.1 | 0.5 |
| 2 | −0.6 | −0.7 | 1.0 | −1.2 | 1.0 | 0.6 | 0.1 | 0.5 |
| 3 | −0.9 | −1.4 | 0.7 | −3.4 | 2.0 | 0.8 | 0.0 | 0.4 |
| 4 | −1.2 | −0.7 | 0.7 | −4.1 | 2.1 | 0.8 | −0.3 | 0.5 |
| 5 | −1.5 | −2.1 | 0.7 | −5.1 | 1.3 | 0.4 | −0.2 | 0.5 |
| 6 | −1.7 | −2.3 | 2.1 | 0.3 | 1.5 | 0.2 | 0.0 | 0.4 |
| 24 | 0.1 | 3.0 | −1.8 | −3.0 | 5.3 | 1.8 | 0.3 | 1.3 |

TABLE 3b
Relative variation (%) of the maximum absorbance
of the π → π * transitions and
charge transfer bands in the UV-Vis spectra of
the complexes 8-11 in 5% dimethyl sulfoxide/95%
Dulbecco's Modified Eagle Medium over 24 h.

| | Complex (λₘₐₓ/nm) | | | | | | | |
|---|---|---|---|---|---|---|---|---|
| Time | 8 | 8 | 9 | 9 | 10 | 10 | 11 | 11 |
| (h) | (292) | (361) | (294) | (375) | (294) | (400) | (292) | (400) |
| 0 | 0.0 | 0.0 | 0.0 | 0.0 | 0.0 | 0.0 | 0.0 | 0.0 |
| 0.25 | −0.1 | −0.3 | −0.2 | −0.7 | 0.0 | −0.8 | −0.2 | −0.1 |
| 0.5 | −0.2 | −0.6 | −0.3 | −0.8 | −1.7 | −0.7 | −0.3 | 0.0 |
| 0.75 | 0.0 | −0.7 | −0.5 | −0.9 | −1.9 | −1.0 | −0.2 | 0.1 |
| 1 | −0.1 | −0.9 | −0.3 | −1.0 | −2.2 | −1.3 | −0.2 | 0.1 |
| 2 | 0.0 | −0.7 | −0.2 | −1.2 | −3.1 | −2.3 | −0.5 | 0.1 |
| 3 | 0.1 | −0.6 | −0.3 | −1.4 | −4.1 | −3.1 | −0.4 | 0.1 |
| 4 | 0.0 | −0.6 | −0.4 | −1.5 | −5.1 | −3.6 | −0.9 | 0.0 |
| 5 | 0.1 | −0.6 | −0.2 | −1.6 | −5.6 | −4.3 | −0.7 | 0.1 |
| 6 | 0.1 | −0.5 | −0.5 | −1.7 | −6.0 | −4.8 | −0.4 | 0.2 |
| 24 | 6.0 | −0.9 | 4.5 | −0.2 | −7.0 | −5.0 | −1.0 | 0.4 |

### Drug Release Studies

### H) Description of the Drug Releasing Studies

By way of example, the release profile of complex 4 from the conjugate RuPC1 in aqueous solution at pH 6.8 and 7.4 was evaluated at room temperature, in dark, over 50 h by analytical RP-HPLC (method 5, as described in section B) Instruments and methods). For each pH value, the chromatograms were acquired at regular periods of time (0 h, 0.75 h, 1.5 h, 2 h, 3 h, 4 h, 5 h, 6 h, 24 h, 50 h) upon rigorous injection of 100 μL of a 0.5 mg/mL solution of RuPC1 in a 1:9 mixture of acetonitrile/phosphate buffer (10 mM, pH 6.8 or 7.4). The compounds detected at each analysis were identified by the retention time values and by ESI-MS of the respective fractions collected (as described in section B) Instruments and methods). The relative (%) area under the curve (AUC) of each compound detected was directly determined in the RP-HPLC software TotalChrom Navigator upon normalization of the chromatograms relative to a blank assay. The percentage release of complex 4 was determined according to equation 1, where % Rₜ corresponds to the percentage release of complex 4 for a given time t; AUCₜ corresponds to the absolute value of the area under the curve of RuPC1 for that given time t determined directly in the software TotalChrom Navigator, and AUCₜₒ corresponds to the initial absolute value of the area under the curve of RuPC1 for t=0 h also determined directly in the software TotalChrom Navigator.

$$\% {R}_{t} = 100 - ( \frac{{AUC}_{t} \times 100}{{AUC}_{{t}_{0}}} ) \qquad Equation 1$$

### I) Results of the Drug Releasing Studies

### Example 23. Release Profile of Complex 4 from the Conjugate RuPC1

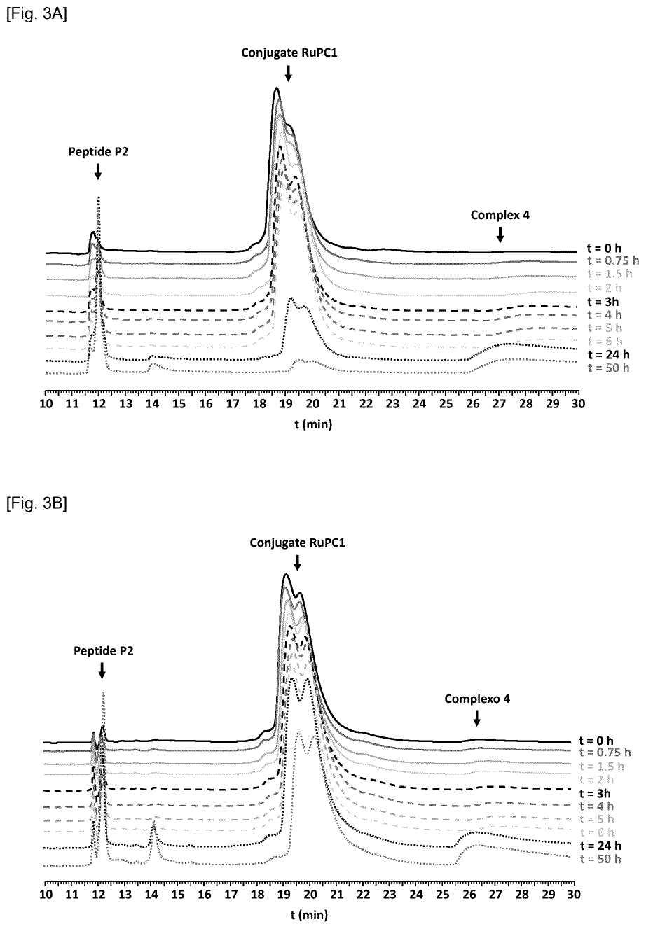

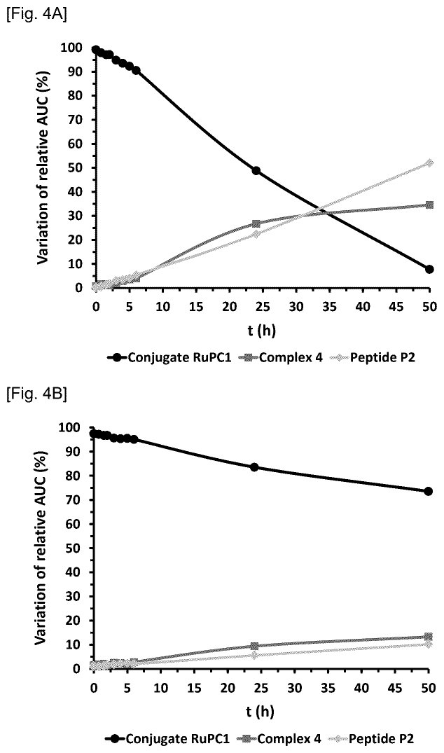

The release profile of complex 4 from the conjugate RuPC1 was determined at pH 6.8, mimic of the tumoral microenvironment, and at pH 7.4, mimic of the bloodstream and healthy tissues. In both cases, the RuPC1 was hydrolyzed over the time, releasing the complex 4 and the peptide P2 (FIGS. 3a, 3b, 4a and 4b). However, the hydrolysis rate of RuPC1 at pH 6.8 is much higher than at pH 7.4. Indeed, after 6, 24 and 50 h it was released 41, 79 and 97% of the complex 2 from the RuPC1 at pH 6.8, respectively, but only 4, 14 and 26% at pH 7.4, respectively (Table 4). These results suggest that RuPC1 has a controlled release profile favorable to the selective delivery of complex 4 into the tumor.

TABLE 4
Percentage release of complex 4 from the conjugate
RuPC1 at pH 6.8 (tumoral microenvironment) and at
pH 7.4 (bloodstream and healthy tissues) over 50 h.

Percentage release of complex 4 (%)

| Time (h) | pH 6.8 | pH 7.4 |
|---|---|---|
| 0 | 0.0 | 0.0 |
| 0.75 | 4.8 | 0.3 |
| 1.5 | 10.5 | 0.4 |
| 2 | 17.5 | 0.3 |
| 3 | 22.6 | 0.2 |
| 4 | 27.7 | 0.6 |
| 5 | 33.5 | 1.2 |
| 6 | 40.9 | 4.0 |
| 24 | 79.1 | 13.5 |
| 50 | 97.3 | 26.8 |

### Biological Studies

### J) Description of the Cytotoxicity Assays

By way of example, the in vitro cytotoxicity of the conjugate RuPC1, complex 4 and peptide P2 was determined in four human breast cancer cell lines with different levels of expression of the receptor FGFR, namely MDA-MB-231 (FGFR−), MCF-7 (FGFR−), SK-BR-3 (FGFR+), and MDA-MB-134-VI (FGFR+). The cytotoxicity of RuPC1 was also determined in the stated cell lines at controlled pH 6.8 and 7.4. The cytotoxicity of the complexes 5, 8, 9, and the reference complex (2,2′-bipyridyl)(triphenylphosphane)(f-cyclopentadienyl)ruthenium(II) trifluoromethanesulfonate, [RuCp(PPh₃)(bipy)][CF₃SO₃] (TM34) was only determined in the cell line MDA-MB-231. All the cell lines were from the American Type Culture Collection (ATCC) and were cultured in Dulbecco's Modified Eagle Medium+Glutamax-I (Gibco) supplemented with 10% fetal bovine serum (FBS) and 1% antibiotics, excepting the MDA-MB-134-VI for which it was used the double amount of FBS. The cell cultures were kept in an incubator Heraeus at 37° C. under humidified atmosphere at 5% CO₂. The cell viability was evaluated resorting to the MTT ([3-(4,5-dimethylthiazol-2-yl)-2,5-diphenyltetrazolium bromide]) colorimetric assay, which is based on the reduction of the tetrazolium salt to purple formazan by mitochondrial dehydrogenases in metabolically active cells. For the assays, the cells were seeded in 96-well plates (2×10⁴ to 3×10⁴ cells/200 μL) and allowed to adhere overnight. The compounds were first dissolved in dimethyl sulfoxide to obtain 10 mM stock solutions, further used to prepare the working solutions, at 0.1 to 100 μM, by dilution in the cell culture medium. Alternatively, for the assays at controlled pH 6.8 and 7.4, RuPC1 was first dissolved in a solution of 15% dimethyl sulfoxide/85% phosphate buffer (10 mM in water) to obtain 1.5 mM stock solutions further incubated for 48 hours at both pH values prior to be used to prepare the working solutions, at 0.1 to 50 μM, by dilution in the cell culture medium. In both cases, after continuous exposure of the cells to the compounds for 48 h at 37° C., the medium was removed and the cells were incubated with 200 μL of MTT solution (0.5 mg/mL) for 3 h. Then, the solution was discarded and the purple formazan formed by the viable cells was dissolved in 200 μL of dimethyl sulfoxide. The cell viability (%) was determined relative to the control (cells incubated without compounds) by measuring the absorbance at 570 nm on a PowerWave Xs Bio-Tek Instruments plate spectrophotometer. The respective values of the half maximal inhibitory concentration (IC₅₀) were calculated with the software GraphPad Prism. All the compounds were tested in at least two independent experiments, each comprising six replicates per concentration.

### K) Results of the Cytotoxicity Assays

### Example 24. In Vitro Cytotoxic Activity of Complexes 4, 5, 8, and 9 in a Triple-Negative Breast Cancer Cell Line Model

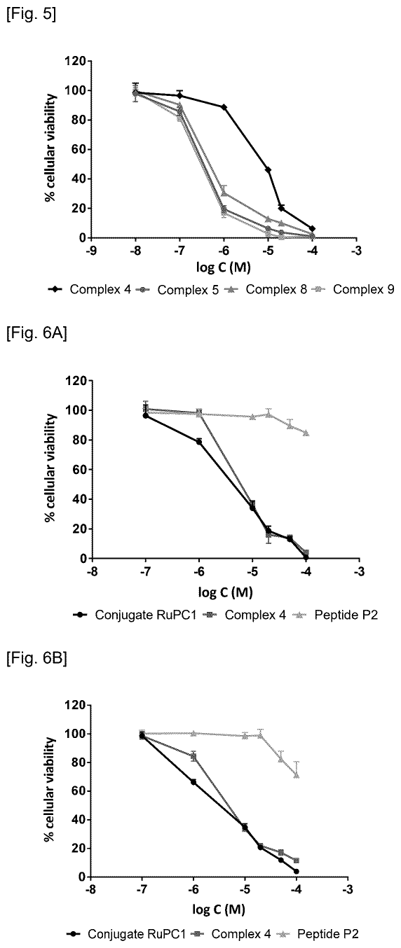

The cytotoxicity of the complexes 4, 5, 8, and 9 was evaluated in vitro in the human triple-negative breast cancer cell line model MDA-MB-231 (FIG. 5). The reference compound [RuCp(PPh₃)(bipy)][CF₃SO₃] (TM34) was also tested for comparison. The results indicate that the ruthenium complexes functionalized with a ketone group at the cyclopentadienyl ring (complexes 5, 8 and 9) kept the high cytotoxicity of TM34, with IC₅₀ values in the sub-micromolar range (Table 5). On the other hand, the derivatization of the cyclopentadienyl ring with a hydrazide group in the complex 4 led to a slightly decrease of the cytotoxicity comparatively to TM34. However, this complex remains highly cytotoxic, with a IC₅₀ value in the low micromolar range (Table 5). Together, the results suggest the suitability of all these complexes to be incorporated in the ruthenium-peptide conjugates sensitive to the tumoral pH.

TABLE 5
Cytotoxic activity expressed as IC₅₀ values (μM) found
for the complexes 4, 5, 8, 9, and TM34 (reference) in the
human breast cancer cell line MDA-MB-231 (48 h, 37° C.).

| Complex | 4 | 5 | 8 | 9 | TM34 |
|---|---|---|---|---|---|
| IC₅₀ (μM) | 7.10 ± | 0.36 ± | 0.56 ± | 0.31 ± | 0.52 ± |
| | 0.96 | 0.07 | 0.12 | 0.05 | 0.14 |

### Example 25. In Vitro Cytotoxic Activity of Conjugate RuPC1, Complex 4, and Peptide P2 in Four Human Breast Cancer Cell Lines with Different Levels of Expression of the Receptor FGFR

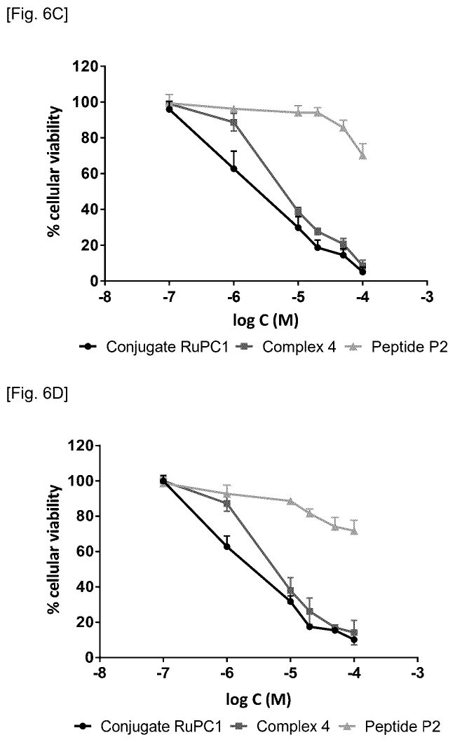

The cytotoxicity of the conjugate RuPC1, complex 4, and peptide P2 was evaluated in vitro in two human breast cancer cell lines that overexpress the receptor FGFR, namely the SK-BR-3 and the MDA-MB-134-VI, and in two another that do not overexpress the receptor, the MDA-MB-231 and the MCF-7 (FIGS. 6A to D). The free peptide did not show significant cytotoxic activity under the adopted experimental conditions (Table 6). On the other hand, the RuPC1 showed to be 1.5- to 2.5-fold more cytotoxic than the complex 4 in all the four cell lines tested, with IC50 values in the low micromolar range (Table 6). Moreover, the conjugate also showed to be 1.5-fold more active in the cell lines that overexpress the FGFR (SK-BR-3 and MDA-MB-134-VI) than in those that do not overexpress (MDA-MB-231 and MCF-7), while the free complex showed the inverse behaviour. These preliminary results confirm the executability on increasing the selectivity of cytotoxic ruthenium complexes towards breast cancer cells that overexpress the FGFR by tethering them to delivering peptides with high affinity for this receptor through a linking moiety sensitive to the tumoral pH.

TABLE 6
Cytotoxic activity expressed as IC₅₀ values (μM) found
for the conjugate RuPC1, complex 4 and peptide P2 in
four human breast cancer cell lines with different levels
of expression of the receptor FGFR (48 h, 37° C.).

| | Human breast cancer cell lines | | | |
|---|---|---|---|---|
| Com- | MDA-MB-231 | MCF-7 | SK-BR-3 | MDA-MB-134-VI |
| pound | (FGFR−) | (FGFR−) | (FGFR+) | (FGFR+) |
| RuPC1 | 4.6 ± 0.7 | 4.0 ± 0.9 | 3.0 ± 1.0 | 3.3 ± 1.0 |
| 4 | 6.1 ± 1.3 | 5.8 ± 0.8 | 7.5 ± 1.0 | 7.1 ± 1.3 |
| P2 | >100 | >100 | >100 | >100 |

### Example 26. In Vitro Cytotoxic Activity of Conjugate RuPC1 in Four Human Breast Cancer Cell Lines with Different Levels of Expression of the Receptor FGFR at Different pH Values

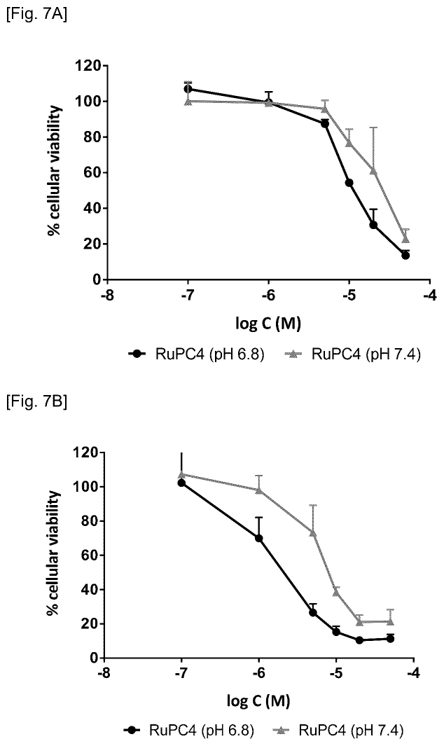

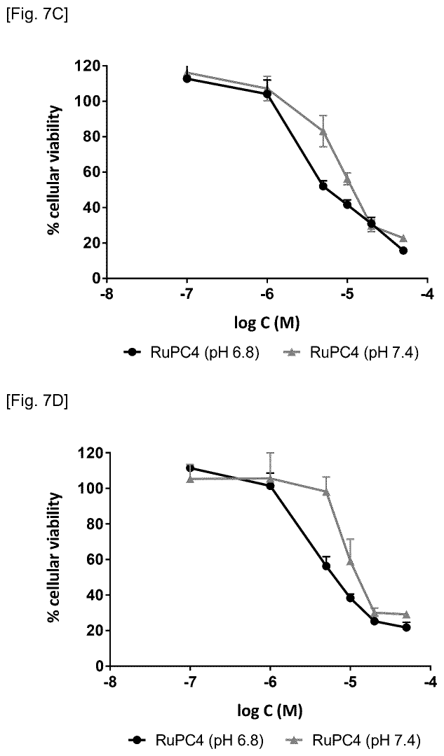

The in vitro cytotoxicity of the conjugate RuPC1 was also evaluated in two human breast cancer cell lines that overexpress the receptor FGFR (SK-BR-3 and MDA-MB-134-VI) and in two another that do not overexpress this receptor (MDA-MB-231 and MCF-7) upon previously incubation of the conjugate for 48 hours in a solution of 15% dimethyl sulfoxide/85% phosphate buffer at pH 6.8 or pH 7.4, to promote the controlled release of complex 4 in its active form (FIGS. 7A to D). In all cell lines, it was observed a higher antiproliferative activity when RuPC1 was previously incubated at the tumor microenvironment's acidic pH (6.8) than when previously incubated at the bloodstream/healthy tissues' pH (7.4), being 2- to 4-fold more cytotoxic in the first case (Table 7). Moreover, RuPC1 once again showed to be more active against the cells overexpressing the receptor FGFR (SK-BR-3 and MDA-MB-134-VI) than against the cell line MDA-MB-231 that does not overexpress this receptor. This data is in good agreement with the release profile of complex 4 from RuPC1 (higher release of the active complex in acidic pH) and suggests that this conjugate not only has tumor-tracking ability (with selectivity to FGFR-overexpressing cancer cells) but also has a prodrug behavior, being more cytotoxic upon activation by the tumoral acidic pH. Altogether with the previous obtained data (examples 23-25), the results also seem to suggest that RuPC1 has the potential to be further studied and eventually developed into a new approach for the precise therapy of metastatic breast cancer.

TABLE 7
Cytotoxic activity expressed as IC₅₀ values (μM)
found for the conjugate RuPC1 at pH 6.8 and pH 7.4
in four human breast cancer cell lines with different
levels of expression of the receptor FGFR (48 h, 37° C.).

| | Human breast cancer cell lines | | | |
|---|---|---|---|---|
| | MDA-MB-231 | MCF-7 | SK-BR-3 | MDA-MB-134-VI |
| pH | (FGFR−) | (FGFR−) | (FGFR+) | (FGFR+) |
| 6.8 | 12.9 ± 3.1 | 2.1 ± 0.7 | 7.9 ± 1.7 | 7.4 ± 1.4 |
| 7.4 | 26.4 ± 9.8 | 8.5 ± 2.3 | 14.0 ± 3.5 | 17.4 ± 5.0 |

The subject matter described above is provided as an illustration of the present invention and, therefore, should not be construed to limit it. The terminology employed for the purpose of describing preferred embodiments of the present invention should not be restricted to them.

As used in the description, defined and indefinite articles, in their singular form, are intended for interpretation to also include plural forms, unless the context of the description explicitly indicates otherwise.

Undefined articles “one” should generally be interpreted as “one or more”, unless the meaning of a singular modality is clearly defined in a specific situation.

It will be understood that the terms “understand” and “include”, when used in this description, specify the presence of characteristics, elements, components, steps and related operations, but do not exclude the possibility of other characteristics, elements, components, steps and operations as well contemplated.

As used throughout this patent application, the term “or” is used in an inclusive sense rather than an exclusive sense, unless the exclusive meaning is clearly defined in a specific situation. In this context, a phrase of the type “X uses A or B” should be interpreted as including all relevant inclusive combinations, for example “X uses A”, “X uses B” and “X uses A and B”.

In addition, the articles “a” and “an” as used in this application and the appended claims should generally be construed to mean “one or more” unless specified otherwise or clear from the context to be directed to a singular form.

All changes, provided they do not modify the essential characteristics of the following claims, must be considered within the scope of the protection of the present invention.

### CITATION LIST

Here follows the list of citations:

### Patent Literature

PTL1: WO 2007/128158 A1
PTL2: WO 2013/136296 A2
PTL3: WO 2016/087932 A2
PTL4: JP 2005047821 A
PTL5: WO 2015081117 A2
PTL6: CN 108484726 A
PTL7: CN 108558950 A

### Non Patent Literature

NPL1: Bruce, M. I.; Windsor, N. J., “Cyclopentadienyl-ruthenium and -osmium chemistry. IV. Convenient high-yield synthesis of some cyclopentadienyl ruthenium or osmium tertiary phosphine halide complexes” Aust. J. Chem. 1977, 30(7), 1601-1604.
NPL2: Franco Machado, J., Machuqueiro, M.; Marques, F.; Robalo, M. P.; Piedade, M. F. M.; Garcia, M. H.; Correia, J. D. G.; Morais, T. S. “Novel “Ruthenium Cyclopentadienyl”-Peptide Conjugate Complexes against Human FGFR(+) Breast Cancer”, Dalton Trans. 2020, 49, 5974.
NPL3: Kaiser, E.; Colescott, R. L.; Bossinger, C. D.; Cook, P. I., “Color test for detection of free terminal amino groups in the solid-phase synthesis of peptides” Anal. Biochem. 1970, 34(2), 595-598.
NPL4: Sand, H.; Weberskirch, R., “Bipyridine-functionalized amphiphilic block copolymers as support materials for the aerobic oxidation of primary alcohols in aqueous media”, RSC Adv. 2015, 5(48), 38235-38242.
NPL5: Splith, K.; Neundorf, I.; Hu, W.; N'Dongo, H. W. P.; Vasylyeva, V.; Merz, K.; Schatzschneider, U. “Influence of the Metal Complex-to-Peptide Linker on the Synthesis and Properties of Bioactive CpMn(CO)₃ Peptide Conjugates”. Dalton Trans. 2010, 39, 2536-2545
NPL6: Hu, W.; Splith, K.; Neundorf, I.; Merz, K.; Schatzschneider, U. “Influence of the Metal Center and Linker on the Intracellular Distribution and Biological Activity of Organometal-Peptide Conjugates”. J. Biol. Inorg. Chem. 2012, 17, 175-185
NPL7: Hoyer, J.; Schatzschneider, U.; Schulz-Siegmund, M.; Neundorf, I. “Dimerization of a Cell-Penetrating Peptide Leads to Enhanced Cellular Uptake and Drug Delivery”. Beilstein J. Org. Chem. 2012, 8, 1788-1797
NPL8: NDongo, H. W. P.; Neundorf, I.; Merz, K.; Schatzschneider, U. “Synthesis, Characterization, X-Ray Crystallography, and Cytotoxicity of a Cymantrene Keto Carboxylic Acid for IR Labelling of Bioactive Peptides on a Solid Support”. J. Inorg. Biochem. 2008, 102, 2114-2119
NPL9: Franco Machado, J.; J. D. G. and Morais, T. S. “Emerging Molecular Receptors for the Specific-Target Delivery of Ruthenium and Gold Complexes into Cancer Cells”. Molecules, 2021, 26, 3153.
NPL10: Jin, Y.; Huang, Y.; Yang, H.; Liu, G.; Zhao, R. “A Peptide-Based pH-Sensitive Drug Delivery System for Targeted Ablation of Cancer Cells”. Chem. Commun. 2015, 51, 14454-14457
NPL11: Zhao, Z.; Zhang, X.; Li, C.; Chen, T. “Designing luminescent ruthenium prodrug for precise cancer therapy and rapid clinical diagnosis”. Biomaterials 2019, 192, 579-589.
NPL12: Zhao, Z.; Gao, P.; You, Y.; Chen, T. “Cancer-Targeting Functionalization of Selenium-Containing Ruthenium Conjugate with Tumor Microenvironment-Responsive Property to Enhance Theranostic Effects”. Chem. Eur. J. 2018, 24, 3289-3298
NPL13: Wang, T.; Zabarska, N.; Wu, Y.; Lamla, M.; Fischer, S.; Monczak, K.; Ng, D. Y. W.; Rau, S.; Weil, T. “Receptor selective ruthenium somatostatin photosensitizer for cancer targeted photodynamic applications”. Chem. Commun. 2015, 51, 12552-12555
NPL14: Lv, G.; Qiu, L.; Li, K.; Liu, Q.; Li, X.; Peng, Y.; Wang, S.; Lin, J. “Enhancement of therapeutic effect in breast cancer with a steroid-conjugated ruthenium complex”. New, J. Chem. 2019, 43, 3419-3427
NPL15: Splith, K.; Hu, W.; Schatzschneider, U.; Gust, R.; Ott, I.; Onambele, L. A.; Prokop, A.; Neundorf, I. “Protease-Activatable Organometal-Peptide Bioconjugates with Enhanced Cytotoxicity on Cancer Cells”. Bioconjug. Chem. 2010, 21, 1288-1296

1. An organometallic complex having the general Formula I 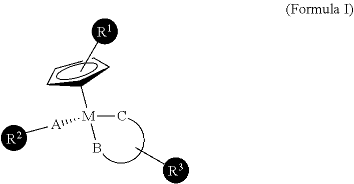
or a pharmaceutically acceptable salt or solvate thereof, wherein:
M is a heteroatom selected from the group consisting of ruthenium, iron, rhodium, cobalt, iridium, osmium, molybdenum, manganese, rhenium, and technetium;
the cyclopentadienyl ring comprises at least one substituent R₁, wherein each substituent R₁ is bonded to any carbon atom of said cyclopentadienyl ring;
A is a monodentate ligand optionally bonded to at least one substituent R₂, the monodentate ligand being preferably selected from the group consisting of heteroaromatic ligands, phosphanes, —CO, —NO, nitriles, isonitriles, a metal complex or a molecule of biological interest;
wherein the heteroatom in the heteroaromatic ligand is selected from the group consisting of O, S, P, N or Se;
wherein the R₂ is present where the rules of valency permit;
wherein the metal complex is selected from the group consisting of inorganic or organometallic complexes with a transition metal selected from the group consisting of ruthenium, iron, rhodium, cobalt, iridium, osmium, molybdenum, manganese, rhenium, and technetium;
wherein the molecule of biological interest is selected from the group consisting of vitamins, hormones, amino acids, sugars, and peptides;
BC together represent a bidentate ligand optionally bonded to at least one substituent R₃, the bidentate ligand being preferably selected from the group consisting of heteroaromatic ligands, diphosphanes, a metal complex or a bidentate molecule of biological interest
wherein the heteroatoms in the heteroaromatic ligands are the same heteroatoms or different heteroatoms, the heteroatoms being independently selected from group consisting of O, S, P, N or Se;
wherein the R₃ is present where the rules of valency permit;
wherein the bidentate molecule of biological interest is selected from the group consisting of vitamins, hormones, amino acids, sugars, and peptides;
wherein BC is taken together with said heteroatom M to which it is attached to form a 3-12-membered heterocyclyl or 5-12 membered heteroaryl ring, wherein each heterocyclyl or heteroaryl ring optionally contains 1, 2 or 3 additional heteroatoms selected from the group consisting of O, S, P, N or Se;
each of R₁, R₂ and R₃ are independently selected from the group consisting of H, C₁-C₆ alkyl, C₁-C₆ aryl, —YCOY′, —YCHO, —YCONHNH₂, —YNHNH₂, —YCOOH, —YCOOY′, —YOCOOH, —YOCOOY′, —YNHCOOH, —YNHCOOY′, —YNHY′, —YONH₂, —YCH(CH₂)ₙ(OH)₂, —YPh(OH)₂, —YSH; —YCH(CH₂)ₙ(SH)₂, —YPh(SH)₂, —YB(OH)₂, —YOB(OH)₂, —YOPO₃H₂, —YOPO₂H₂, —Y(CH₂)ₙX, —YCOX, —YCHCH₂, —YN₃, —Y-imidazole, —YOCOO(CH₂)₂SH, —YNHCOO(CH₂)₂SH and —Y-AA,
wherein
Y and Y′ are independently selected from the group consisting of H, C₁-C₆ alkyl and C₁-C₆ aryl; each of said alkyl or aryl being unsubstituted or substituted by carbonyl, amide, ester, ether or thioether;
n is an integer selected from 0 to 6;
Ph is a phenyl group;
X is a halogen atom selected from the group consisting of F, Cl, Br and I;
the heteroaryl ring is a 5-12 membered heteroaryl ring wherein each heteroaryl ring optionally contains 1, 2 or 3 heteroatoms selected from the group consisting of O, S, P, N or Se; and
AA is a cis-aconitic anhydride group;
with the proviso that at least one of R₁, R₂ or R₃ is not H.

2. The organometallic complex or a pharmaceutically acceptable salt or solvate thereof, according to claim 1, wherein:
M is ruthenium;
the cyclopentadienyl ring comprises one substituent R₁, wherein such substituent R₁ is bonded to any carbon atom of said cyclopentadienyl ring;
A is a monodentate ligand;
BC is a bidentate heteroaromatic ligand wherein both heteroatoms are nitrogen;
each of R₁, R₂ and P3 are independently selected from the group consisting of H, C₁-C₆ aryl, —YCOY′, —YCONHNH₂ and —YNHNH₂
wherein
Y and Y′ are independently selected from the group consisting of H, C₁-C₆ alkyl and C₁-C₆ aryl; each of said alkyl or aryl being unsubstituted or substituted by carbonyl, amide, ester, ether or thioether; and
the heteroaryl ring is a 5-12 membered heteroaryl ring wherein each heteroaryl ring optionally contains 1, 2 or 3 heteroatoms selected from the group consisting of O, S, P, N or Se; and
with the proviso that at least one of R₁, R₂ or R₃ is not H.

3. The organometallic complex or a pharmaceutically acceptable salt or solvate thereof, according to claim 1, wherein:
M is ruthenium;
the cyclopentadienyl ring comprises one substituent R₁, wherein such substituent R₁ is bonded to any carbon atom of said cyclopentadienyl ring;
A is triphenylphosphane and R₂ is H;
BC is 2,2′-bipyridyl; 1,10-phenanthroline or pyrazino[2,3-f][1,10]phenanthroline;
each of R₁ and R₃ are independently selected from the group consisting of —H, —COCH₃, —COCH₂CH₃, —COCH₂Ph, —CONHNH₂, —OH, —OCO₂CH₂CH₃ and —OCO(CH₂)₂COCH₃;
with the proviso that at least one of R₁ or P3 is not H.

4. The organometallic complex, according to claim 1, wherein it is chlorobis(triphenylphosphane)(η⁵-methyl cyclopentadienylketone)ruthenium(II);
chlorobis(triphenylphosphane)(η⁵-ethyl cyclopentadienylketone) ruthenium(II);
chlorobis(triphenylphosphane)(η⁵-phenylmethyl cyclopentadienylketone) ruthenium(II);
(2,2′-bipyridyl)(triphenylphosphane)(Us-cyclopentadienylhydrazide) ruthenium(II) trifluoromethanesulfonate;
(2,2′-bipyridyl)(triphenylphosphane)(η⁵-methyl cyclopentadienylketone) ruthenium(II) trifluoromethanesulfonate;
(1,10-phenanthroline)(triphenylphosphane)(η⁵-methyl cyclopentadienylketone) ruthenium(II) trifluoromethanesulfonate;
(pyrazino[2,3-f][1,10]phenanthroline)(triphenylphosphane)(η⁵-methyl cyclopentadienylketone) ruthenium(II) trifluoromethanesulfonate;
(2,2′-bipyridyl)(triphenylphosphane)(η⁵-ethyl cyclopentadienylketone) ruthenium(II) trifluoromethanesulfonate;
(2,2′-bipyridyl)(triphenylphosphane)(η⁵-phenylmethyl cyclopentadienylketone) ruthenium(II) trifluoromethanesulfonate;
(4-hydroxy-4′-methoxy-2,2′-bipyridyl)(triphenylphosphane)(η⁵-cyclopentadienyl) ruthenium(II) trifluoromethanesulfonate;
(4-(ethoxycarbonyl)oxy-4′-methoxy-2,2′-bipyridyl)(triphenylphosphane)(η⁵-cyclopentadienyl) ruthenium(II) trifluoromethanesulfonate; or
(4-methoxy-4′-(4-oxopentanoate)-2,2′-bipyridyl)(triphenylphosphane)(η⁵-cyclopentadienyl) ruthenium(II)trifluoromethanesulfonate.

5. A process for the preparation of the organometallic complex according to claim 1, characterized by
reacting a compound of formula [M(Cp-R₁)(LL)X] or its solvates or its salts with A-R₂ or BC-R₃ ligands in the presence or in the absence of a halide abstraction reagent selected from the group consisting of AgCF₃SO₃, AgBF₄, AgPF₆, AgNO₂, AgNO₃, AgSO₄, NaCF₃SO₃, NaBF₄, NaPF₆, NaBArF, KCF₃SO₃, NH₄CF₃SO₃ or TlCF₃SO₃,
wherein Cp is a cyclopentadienyl ring,
wherein LL represents two A-R₂ ligands; one BC-R₃ ligand; two monodentate ligands; or one bidentate ligand, and
wherein X is a halide selected from the group consisting of fluoride, chloride, bromide, or iodide.

6.-13. (canceled)

14. A process for the preparation of the organometallic complex according to claim 1, characterized by reacting a compound of formula [M(Cp-F₁)(A-F₂)(BC-F₃)] or its solvates or its salts with a reactant or group of reactants to obtain the organometallic complex by addition, elimination, substitution, abstraction, conjugation, coupling, condensation, esterification, amidation, amination, alkylation, rearrangement, metathesis, cyclization, pericyclization, cycloaddition, hydrolysis, hydrogenation, redox, metalation, or any other suitable chemical reaction,
wherein Cp is a cyclopentadienyl ring, and
wherein F₁, F₂, and F₃ independently represent a chemical group which upon reaction, functionalization, or modification can be converted to R₁, R₂, and R₃, respectively.

15. The process for the preparation of the organometallic complex, according to claim 5, wherein the chemical reaction occurs in a solvent or a mixture of solvents that allow and/or facilitate said reaction, the solvent or mixture of solvents being selected from the group consisting of water, acetone, toluene, hexane, methanol, tetrahydrofuran, pyridine, dichloromethane, dioxane, chloroform, ethanol, acetic acid, carbon tetrachloride, tert-butanol, dimethylglyoxime, trifluoroacetic acid, ethylene glycol, cyclohexane, dichloroethane, isopropanol, pentane, diethyl ether, propanol, dimethylformamide, benzene, dimethyl sulfoxide, ethyl acetate, xylene, butanol, dioxane, or heptane.

16. The process for the preparation of the organometallic complex, according to claim 5, wherein the temperature ranges from −20° C. to 150° C. and the pressure ranges from 0.1 and 10 atm; with or without stirring; under atmosphere of inert gas selected from dinitrogen or argon and in the presence or in the absence of irradiation by ultrasounds, microwaves, infrared light, ultraviolet light or visible light; and with or without the addition of a salt, acid, base, catalyst or coupling reagent.

17. The process for the preparation of the organometallic complex, according to claim 5, wherein the products are obtained without further purification or isolated and further purified by any suitable analytical method selected from the group consisting of crystallization, recrystallization, co-crystallization, sublimation, distillation, chromatography, filtration, precipitation, or extraction.

18. An organometallic complex or a pharmaceutically acceptable salt or solvate thereof according to claim 1, wherein it is for use, alone or in combination with an approved drug and/or pharmaceutical product, as an antitumoral agent and/or anti-metastatic and/or as a radio sensitizer agent in cancer therapy and/or cancer prevention or it is for use as a diagnosis agent and/or theragnostic agent in cancer identification, independently of the stage of the disease.

19. (canceled)

20. The organometallic complex or a pharmaceutically acceptable salt or solvate thereof for use, according to claim 18, wherein cancer is either primary cancers and/or cancer metastasis, selected from the group consisting of breast cancer, lymphoma, skin cancer, ovarian cancer, liver cancer, cervix cancer, head and neck cancer, colon cancer, testicular cancer, larynx cancer, nasopharynx cancer, bladder cancer, oropharynx cancer, lung cancer, rectal cancer, brain cancer, prostate cancer, stomach cancer, kidney cancer, esophageal cancer, thyroid cancer, pancreatic cancer, bone cancer, or leukemia.

21. The organometallic complex or a pharmaceutically acceptable salt or solvate thereof for use, according to claim 18, wherein it is for topical, intravenous, subcutaneous or intraperitoneal administration.

22. A controlled-release multi-functional drug characterized by having the general Formula II
OC-L-TU (Formula II)
wherein
OC is the organometallic complex according to claim 1, or a pharmaceutically acceptable salt or solvate thereof,
TU is a targeting unit selected from the group consisting of peptide, protein, amino acid, peptidomimetic, antibody, affibody, nanobody, aptamer, antigen, or binding fragments thereof, which are able to selectively bind to receptors or drug targets overexpressed at cancer cell surfaces,
wherein the targeting unit TU has a R₄ group in its structure, the R₄ group being selected from the group consisting of H, C₁-C₆ alkyl, C₁-C₆ aryl, —YCOY′, —YCHO, —YCONHNH₂, —YNHNH₂, —YCOOH, —YCOOY′, —YOCOOH, —YOCOOY′, —YNHCOOH, —YNHCOOY′, —YNHY′, —YONH₂, —YCH(CH₂)ₙ(OH)₂, —YPh(OH)₂, —YSH; —YCH(CH₂)ₙ(SH)₂, —YPh(SH)₂, —YB(OH)₂, —YOB(OH)₂, —YOPO₃H₂, —YOPO₂H₂, —Y(CH₂)ₙX, —YCOX, —YCHCH₂, —YN₃, —Y-imidazole, —YOCOO(CH₂)₂SH, —YNHCOO(CH₂)₂SH and —Y-AA,
wherein
Y and Y′ are independently selected from the group consisting of H, C₁-C₆ alkyl and C₁-C₆ aryl; each of said alkyl or aryl being unsubstituted or substituted by carbonyl, amide, ester, ether or thioether;
n is an integer selected from 0 to 6;
Ph is a phenyl group;
X is a halogen atom selected from the group consisting of F, Cl, Br and I;
the heteroaryl ring is a 5-12 membered heteroaryl ring wherein each heteroaryl ring optionally contains 1, 2 or 3 heteroatoms selected from the group consisting of oxygen, sulfur, phosphorus, nitrogen and selenium; and
AA is a cis-aconitic anhydride group;
the organometallic complex OC and the targeting unit TU being bonded via a linking moiety L between the organometallic complex OC and the targeting unit TU;
wherein the linking moiety L results from the reaction of the R₄ from the targeting unit TU with the R′ from the organometallic complex OC;
wherein the R′ from organometallic complex OC is any one of R₁, R₂ or R₃;
wherein at least one linking moiety L and at least one targeting unit TU are bonded in the same R′ or in different R′;
wherein the linking moiety L is selected from the group consisting of —YC(Y′)═NNH—; —YNHN═C(Y′)—; —YC(Y′)═NNHC(O)—; —YC(O)NHN═C(Y′)—; —YC(Y′)═NC—; —C(Y′)═NCY—; —YPhC(Y′)═NC—; —YC(Y′)═NPh-; —YN═NY′—; —YC(O)NY′—; —C(O)N(Y′)Y—; cis-aconityl; —YC(Y′)═NO—; —C(Y′)═NOY—; —YC(Y′)(OY″)O—; —YC(Y′)(OH)O—; —YC(O)OC—; —YC(OY′)(OY″)OC—; —YOB(OY′)OC—; —YB(OY′)OC—; —YOC(O)OC—; bis-carbonate; para-aminobenzylcarbonate; —YOC(O)NY′—; —OC(O)N(Y′)Y—; para-methoxybenzyl carbamate, —YOC(O)CH₂CH₂SY′—; N-ethoxybenzylimidazole; N-alkoxyalkylimidazole; phosphoramidyl; phosphoramidate; —YSSY′—; disulphide carbamate, —YC(Y′)(SY″)S—; bis-alkylcarbonate dissulphide; bis-alkylcarbonate thioketal; —YSY′—; β-glucuronide; β-galactoside; or trityl,
wherein
Y, Y′ and Y″ are independently selected from the group consisting of H, C₁-C₆ alkyl and C₁-C₆ aryl; each of said alkyl or aryl being unsubstituted or substituted by carbonyl, amide, ester, ether or thioether;
n is an integer selected from 0 to 6;
Ph is a phenyl group; and
the heteroaryl ring is a 5-12 membered heteroaryl ring wherein each heteroaryl ring optionally contains 1, 2 or 3 heteroatoms selected from the group consisting of oxygen, sulfur, phosphorus, nitrogen and selenium.

23. The controlled-release multi-functional drug, according to claim 22, wherein
OC is the organometallic complex according to claim 1, or a pharmaceutically acceptable salt or solvate thereof,
TU is a peptide having a R₄ group in one of its end, the R₄ group being selected from the group consisting of H, CO(CH₂)₂COCH₃, CO(CH₂)₂CONHNH₂ and CO(CH₂)₂CO₂CO(CH₂)₂CONHNH₂.
the organometallic complex OC and the targeting unit TU being bonded via a linking moiety L between the organometallic complex OC and the targeting unit TU;
wherein the linking moiety L has a hydrolytically cleavable bond and is selected from the group consisting of CONHNC(CH₃)(CH₂)₂CO, C(CH₃)NNHCO(CH₂)₂CO, C(CH₃)NNHCO(CH₂)₂CO, C(CH₂CH₃)NNHCO(CH₂)₂CO and C(CH₂Ph)NNHCO(CH₂)₂CO.

24. The controlled-release multi-functional drug, according to claim 22, wherein it is:
(2,2′-bipyridyl)(triphenylphosphane)(4-((E,Z)-2-η⁵-cyclopentadienylformylhydrazineylidene)pentanoyl-VSPPLTLGQLLS)ruthenium(II) trifluoromethanesulfonate;
(2,2′-bipyridyl)(triphenylphosphane)(4-((E,Z)-2-(1-η⁵-cyclopentadienylethylidene)hydrazineyl)-4-oxobutanoyl-VSPPLTLGQLLS)ruthenium(II) trifluoromethanesulfonate; and
(1,10-phenanthroline)(triphenylphosphane)(4-((E,Z)-2-(1-η⁵-cyclopentadienylethylidene)hydrazineyl)-4-oxobutanoyl-VSPPLTLGQLLS)ruthenium(II) trifluoromethanesulfonate.

25. A process for the preparation of the controlled-release multi-functional drug according to claim 22, characterized by
reacting the organometallic complex according to claim 1 with one or more targeting units containing a R₄ group,
wherein the R₁, R₂ and/or R₃ groups of the organometallic complex is reacted with the R₄ by means of conjugation, coupling, cross-coupling, condensation, rearrangement, addition, elimination, substitution, cycloaddition, redox, or any other suitable chemical reaction in a solvent or a mixture of solvents that allow and/or facilitate said reaction, in the presence or the absence of a catalyst or coupling agent.

26. The process for the preparation of the controlled-release multi-functional drug, according to claim 25, wherein the solvent or mixture of solvents is selected from the group consisting of water, acetone, toluene, hexane, methanol, tetrahydrofuran, pyridine, dichloromethane, dioxane, chloroform, ethanol, acetic acid, carbon tetrachloride, tert-butanol, dimethylglyoxime, trifluoroacetic acid, ethylene glycol, cyclohexane, dichloroethane, isopropanol, pentane, diethyl ether, propanol, dimethylformamide, benzene, dimethyl sulfoxide, ethyl acetate, xylene, butanol, dioxane, or heptane.

27. The process for the preparation of the controlled-release multi-functional drug, according to claim 25, wherein the temperature ranges from −20° C. to 150° C. and the pressure ranges from 0.1 and 10 atm; with or without stirring; under atmosphere of inert gas selected from dinitrogen or argon and in the presence or in the absence of irradiation by ultrasounds, microwaves, infrared light, ultraviolet light or visible light; and with or without the addition of a salt, acid, base, catalyst or coupling reagent.

28. The process for the preparation of the controlled-release multi-functional drug, according to claim 25, wherein the products are obtained without further purification or isolated and further purified by any suitable analytical method selected from the group consisting of crystallization, recrystallization, co-crystallization, sublimation, distillation, chromatography, filtration, precipitation, or extraction.

29. A controlled-release multi-functional drug or a pharmaceutically acceptable salt or solvate thereof according to claim 22, wherein it is for use, alone or in combination with an approved drug and/or pharmaceutical product, as an antitumoral agent and/or anti-metastatic agent and/or as a radio sensitizer agent in cancer therapy and/or cancer prevention or it is for use as a diagnosis agent and/or theragnostic agent in cancer identification, independently of the stage of the disease.

30. (canceled)

31. The controlled-release multi-functional drug or a pharmaceutically acceptable salt or solvate thereof for use, according to claim 29, wherein the cancer is either primary cancers and/or cancer metastasis, selected from the group consisting of breast cancer, lymphoma, skin cancer, ovarian cancer, liver cancer, cervix cancer, head and neck cancer, colon cancer, testicular cancer, larynx cancer, nasopharynx cancer, bladder cancer, oropharynx cancer, lung cancer, rectal cancer, brain cancer, prostate cancer, stomach cancer, kidney cancer, esophageal cancer, thyroid cancer, pancreatic cancer, bone cancer, or leukemia.

32. The controlled-release multi-functional drug or a pharmaceutically acceptable salt or solvate thereof for use, according to claim 29, wherein it is for topical, intravenous, subcutaneous or intraperitoneal administration.

33. A pharmaceutical composition characterized by comprising a pharmaceutically effective amount of an organometallic complex according to claim 1, or a pharmacologically acceptable salt and/or solvate thereof, or a controlled-release multi-functional drug according to claim 22 or a pharmaceutically acceptable salt or solvate thereof and at least one active ingredient and/or pharmaceutically acceptable carrier, excipient, or diluent.

34. The pharmaceutical composition, according to claim 33, wherein it is for use, alone or in combination with an approved drug and/or pharmaceutical product, as an antitumoral agent and/or anti-metastatic agents and/or as a radio sensitizer agent in cancer therapy and/or cancer prevention, or it is for use as a diagnosis agent and/or theragnostic agent in cancer identification, independently of the stage of the disease.

35. (canceled)

36. The pharmaceutical composition for use; according to claim 33, wherein the cancer is either primary cancers and/or cancer metastasis, selected from the group consisting of breast cancer, lymphoma, skin cancer, ovarian cancer, liver cancer, cervix cancer, head and neck cancer, colon cancer, testicular cancer, larynx cancer, nasopharynx cancer, bladder cancer, oropharynx cancer, lung cancer, rectal cancer, brain cancer, prostate cancer, stomach cancer, kidney cancer, esophageal cancer, thyroid cancer, pancreatic cancer, bone cancer, or leukemia.

37. The pharmaceutical composition for use according to claim 33, wherein it is for topical, intravenous, subcutaneous or intraperitoneal administration.
